(19) United States
(12) Patent Application Publication
(10) Pub. No.: US 2010/0285000 A1
(43) Pub. Date: Nov. 11, 2010

(54) USE OF VEGFR-2 INHIBITORS FOR TREATING METASTATIC CANCER

(75) Inventor: Roni Mamluk, Mazkeret Batia (IL)

Correspondence Address: ROPES & GRAY LLP, PATENT DOCKETING Floor 39, One International Place, Boston, MA, 02110-2624, US

(73) Assignee: BRISTOL-MYERS SQUIBB COMPANY, Princeton, NJ (US)

(21) Appl. No.: 12/674,144

(22) PCT Filed: Aug. 20, 2008
(86) PCT No.: PCT/US2008/009890

§ 371 Date: Jul. 14, 2010

Related U.S. Application Data
(60) Provisional application No. 60/965,574, filed on Aug. 20, 2007.

(51) Int. Cl. A61K 39/395; A61K 38/18; A61K 35/00

(52) U.S. Cl. 424/130.1; 514/8.1

(57) ABSTRACT

The present application provides compositions and methods for treating metastatic cancer. Patients having or at risk of developing metastases may be treated. Compositions useful for the invention include VEGFR-2 specific inhibitors.

### CROSS REFERENCE TO RELATED APPLICATION

This application claims the benefit of U.S. Provisional Application Ser. No. 60/965,574 filed Aug. 20, 2007, the entire contents of which are incorporated herein by reference.

### FIELD OF THE INVENTION

The present invention relates to the treatment of metastatic cancer using innovative proteins that block the VEGF-VEGFR pathyway mediated biology and pathology. The invention also relates to innovative proteins in pharmaceutical preparations and derivatives of such proteins and the uses of same in the treatment of metastatic cancer.

Introduction

The formation of metastases of malignant tumors, initiated from a primary tumor at more or less remote locations of the body, is one of the most serious effects of cancer and one for which a satisfactory treatment protocol is currently unavailable. Cancer tumor metastasis is responsible for most therapeutic failures when the disease is treated, as patients succumb to the multiple tumor growth.

The extent to which metastases occur vary with the individual type of tumor. Melanoma, lymphoma, breast cancer, lung cancer, colon cancer and prostate cancer are among the types of cancers that are particularly prone to metastasize. When metastasis takes place, the secondary tumors can form at a variety of sites in the body, with lungs, liver, brain and bone being the more common sites.

The currently available methods of cancer therapy such as surgical therapy, radiotherapy, chemotherapy and other immunobiological methods have either been unsuccessful in preventing metastasis or these methods give rise to serious and undesirable side effects.

In many clinically diagnosed solid tumors (in which the tumor is a localized growth) surgical removal is considered the prime means of treatment. However, many times after surgery and/or after some delay period, the original tumor is observed to have metastasized so that secondary sites of cancer invasion have spread throughout the body and the patient subsequently dies of the secondary cancer growth. In other embodiments surgical removal of the tumor is not feasible because of the location of the tumor (for example certain areas in the brain) and radiation, chemotherapy or other immunobiological methods are the sole alternatives.

Reports indicate that in individuals with resectable tumors, primary tumor growth or local recurrence is not often the cause of death. Instead, at present, nearly 40% of cancer victims with operable tumors ultimately succumb to metastatic disease following surgery.

Metastasis is a constant occurrence in some tumors. However, many times metastasis is triggered by the surgery itself. During the course of surgery malignant cells may become dislodged from the tumor mass and enter the circulatory system thus increasing the chance of metastasis.

Although chemotherapy is widely used in the treatment of cancer, it is a systemic treatment based usually on the prevention of cell proliferation. Accordingly, chemotherapy is a non-specific treatment modality affecting all proliferating cells, including normal cells, leading to undesirable and often serious side effects such as immunosuppression, pancytopenia (growth inhibition of bone marrow cells with anemia, thrombocytopenia, and leukopenia), diarrhea, nausea or alopecia (hair loss).

Generally, the existing systemic treatments have, quite often, proven to have little effect on micrometastases already residing in remote organs (lung, liver, bone marrow or brain), and they are not very effective in preventing the dissemination of the tumor to other tissues.

Therefore, the need exists for methods for inhibiting tumor metastasis. In particular, methods which inhibit (micro)metastasis without causing serious side effects are much desired.

### SUMMARY OF THE INVENTION

One aspect of the invention provides methods for the treatment of a subject having or at risk of developing metastatic cancer by administering a novel protein of the present invention, either alone or in combination with other cytotoxic or therapeutic agents. The cancer can be one or more of, for example, breast cancer, colon cancer, ovarian carcinoma, osteosarcoma, cervical cancer, prostate cancer, lung cancer, synovial carcinoma, melanoma, skin, pancreatic cancer, or other cancer yet to be determined in which VEGFR-2 levels are elevated, up-regulated, mutated or altered in physiology compared to non-oncogenic cells.

The invention provides methods for the treatment of a subject having or at risk of developing metastatic cancer by administering an novel protein of the present invention, either alone or in combination with other cytotoxic or therapeutic agents. In particular, preferred cytotoxic and therapeutic agents include docetaxel, paclitaxel, doxorubicin, epirubicin, cyclophosphamide, trastuzumab (Herceptin™), capecitabine, tamoxifen, toremifene, letrozole, anastrozole, fulvestrant, exemestane, goserelin, oxaliplatin, carboplatin, cisplatin, dexamethasone, antide, bevacizumab (bevacizumab™), 5-fluorouracil, leucovorin, levamisole, irinotecan, etoposide, topotecan, gemcitabine, vinorelbine, estramustine, mitoxantrone, abarelix, zoledronate, streptozocin, rituximab (Rituxan™), idarubicin, busulfan, chlorambucil, fludarabine, imatinib, cytarabine, ibritumomab (Zevalin™), tositumomab (Bexxar™), interferon alpha-2b, melphalam, bortezomib (Velcade™), altretamine, asparaginase, gefitinib (Iressa™), erlonitib (Tarceva™), anti-EGF receptor antibody (Cetuximab™, Abx-EGF), and an epothilone. More preferably, the therapeutic agent is a platinum agent (such as carboplatin, oxaliplatin, cisplatin), a taxane (such as paclitaxel, docetaxel), gemcitabine, or camptothecin.

In one embodiment, a method of treating a medical condition is provided, comprising, administrating a protein of the invention. In a further embodiment, the protein administered is an anti-angiogenesis agent. In another embodiment, a second agent is also administered. The second agent may be any of the cytotoxic or therapeutic agents herein disclosed and by way of example can be selected from the following list of therapeutic agents: Sutent™ (i.e., sunitinib malate, described in U.S. Pat. No. 6,573,293), Tykreb™ (i.e., lapatinib, described in U.S. Pat. No. 6,727,256), Nexavar™ (i.e., Bayer BAY 43-9006/sorafenib, described in U.S. patent application Ser. No. 09/425,228 and PCT/US00/00648), AZD2171 (i.e., Recentin™, disclosed in PCT International Application Publication No. WO 00/47212), bevacizumab (i.e., bevacizumab™, described in U.S. Pat. No. 6,054,297), VEGF Trap (such as described in Kim et al. (2002) Proc. Natl. Acad. Sci. USA 99:11399-404; Holash et al. (2002) Proc. Natl. Acad. Sci. USA 99:11393-8 and by example AVE0005), mTor inihibitors such as rapamycin and its derivatives (the preparation and use of hydroxyesters of rapamycin, including CCl-779, i.e., temsirolimus, are disclosed in U.S. Pat. No. 5,362,718), kinase inihibitors that act on a cytosolic portion of said kinase, gemcitabine (i.e., Gemzar™, disclosed in U.S. Pat. No. 4,808,614), temozolomide (i.e., Temodar™, the use of which in treating cancer is disclosed in U.S. Pat. No. 5,939,098), dasatinib (i.e., Sprycel™, disclosed in U.S. Pat. No. 6,596,746), cetuximab (Erbitux™, disclosed in, for example, U.S. Pat. No. 6,217,866), ixabepilone (Bristol-Myers Squibb's BMS-247550), imatinib mesylate (i.e., Gleevac™, disclosed in U.S. Pat. No. 5,521,184), trastuzumab (Herceptin™, disclosed in, for example, U.S. Pat. No. 5,677,171), and members of the taxane class such as Paclitaxel (i.e., Taxol™, disclosed in U.S. Pat. No. 5,439,686) and docetaxel (i.e., Taxotere™, disclosed in U.S. Pat. No. 4,814,470). In one embodiment, the protein of the invention is administered once every two weeks. In one embodiment, the method of treating a medical condition comprises administering to a subject a therapeutically effective amount of a protein of the invention. In another embodiment, the method of treatment further comprises administering to a subject a therapeutically effective amount of a second therapeutic agent. In one aspect the subject treated is a human.

In addition, it may be preferred to combine a protein of the invention with a second therapeutic protein as a single molecule or perhaps as a single molecule with a third therapeutic protein. Such a therapeutic entity, comprises a protein of the invention to VEGFR-2 linked by PEG or other polymer (e.g., Cys-Cys disulfide or polypeptide) to one or more therapeutic proteins. Such therapeutic proteins include antibody derivatives (e.g., Fabs, camel antibodies and their derivatives, domain antibodies (e.g., less than about 50 kD in size) and single chains (preferably less than about 50 kD in size)), fibronectin based scaffolds, such as an Adnectins™ and proteins preferably in the range of ˜5 to ˜40 kd.

Targets of proteins of the invention include, particularly human versions, although in some instances model species such as mouse, rat, monkey and dog: FGFR1, FGFR2, FGFR3, FGFR4, c-Kit, human p185 receptor-like tyrosine kinase, HER2/Her2, Her3, c-Met, folate receptor, PDGFR, VEGFR1, VEGFR2, VEGFR3, human vascular endothelial growth factor (VEGF) A, VEGF C, VEGF D, human CD20, human CD18, human CD11a, human apoptosis receptor-2 (Apo-2), human .alpha.4.beta.7 integrin, human GPIIb-IIIa integrin, stem cell factor (SCF), human epidermal growth factor receptor (EGFR), and human CD3. In addition, aspects of the invention include multifunctional proteins that bind a first target and at least one other target. Preferably, such proteins are linked by the PEG related inventions described herein, although in many embodiments such proteins may be linked by polypeptides or other polymeric linkers or non-polymeric linkers.

Stably linked proteins of the invention may be of use for therapeutic treatment of cancer. Multispecific proteins of the invention have the advantage of modulating, blocking or inhibiting more than one therapeutic target when directed to 2, 3, 4 or more therapeutic targets or epitopes.

It is anticipated that any type of tumor and any type of tumor antigen may be targeted with the corresponding biology of the therapeutic. The cancer can be one or more of, for example, breast cancer, colon cancer, ovarian carcinoma, osteosarcoma, cervical cancer, prostate cancer, lung cancer, synovial carcinoma, pancreatic cancer, melanoma, multiple myeloma, neuroblastoma, and rhabdomyosarcoma, or other cancer yet to be determined in which VEGFR-2 levels are elevated, up-regulated, mutated or altered in physiology compared to non-oncogenic cells.

Other exemplary types of tumors that may be targeted include acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer.

In some embodiments a method of treating, preventing, or reducing the spread of metastatic cancer is provided, the method comprising administering, to a patient in need thereof, a therapeutic VEGFR-2 specific inhibitor. In some embodiments, the patient is afflicted with breast cancer. In some embodiments, the VEGFR-2 specific inhibitor comprises a first polypeptide that binds to human VEGFR-2, the polypeptide comprising between about 80 and about 150 amino acids that have a structural organization comprising at least five to seven beta strands or beta-like strands distributed among at least two beta sheets, and at least one loop portion connecting two strands that are beta strands or beta-like strands, which loop portion participates in binding to VEGFR-2, wherein the polypeptide binds to an extracellular domain of the human VEGFR-2 protein with a dissociation constant (K_{d}) of less than 1×10⁻⁶ M and inhibits VEGFR-2 mediated angiogenesis. In some embodiments, the polypeptide comprises an amino acid sequence that is at least 80% identical to SEQ NO:1. In some embodiments, the polypeptide comprises an amino acid sequence selected from the group consisting of any of SEQ ID NOs:2-60. In some embodiments, the polypeptide further comprises a polyoxyalkylene moiety. In some embodiments, the polyoxyalkylene moiety is a polyethylene glycol (PEG) moiety.

In some embodiments, the methods of treatment comprise the administration of a VEGFR-2 specific inhibitor and a second chemotherapeutic agent. In some embodiments, the second agent is selected from sunitinib malate, lapatinib, sorafenib, AZD2171, bevacizumab, aflibercept, an mTor inhibitor, rapamycin, gemcitabine, temozolomide, dastinib, cetuximab, temsirolimus, ixabepilone, imatinib mesylate, trastuzumab, a taxane, oxaliplatin, 5-fluorouracil. In some embodiments, the VEGFR-2 specific inhibitor further comprises a second polypeptide, the polypeptide comprising an amino acid sequence that is at least 80% identical to SEQ NO: 1.

The VEGFR-2 specific inhibitors useful for the methods include small molecules, polymers, sugars and other macromolecules, polypeptides (including antibodies), or nucleic acids (including antisense nucleic acids, ribozymes, and small interfering RNAs or siRNAs). A VEGFR-2 specific inhibitor encompasses any composition that modulates, affects, alters, inhibits or reduces the activity of VEGFR-2, including target binding, enzymatic activity or tyrosine phosphorylation action of a tyrosine kinase, preferably by at least 5, 10, 20, 30, 40, 50, 60, 70, 80, 90, 95, 97, 98, 99 or 100%. In exemplary embodiments, VEGFR-2 specific inhibitors are proteins.

It will be often desirable, particularly in in vivo applications, that the VEGFR-2 specific inhibitors are selective over VEGFR-2 compared to VEGFR-1 and VEGFR-3. Such selectivity is preferably at least about 100 times, at least about 1000 times, at least about 10,000 times and at least about 100,000 times. In addition, it my be desirable, particularly for inhibitors with high affinity VEGFR-2 to not detectably bind VEGFR-1 and VEGFR-3 at a defined concentration or lower of therapeutic or protein, such concentrations are about 100 nM, about 1 μM, or about 10 μM. Selectivity relationships of inhibitors that bind to VEGFR-2 over VEGFR-1 and VEGFR-3 may also be expressed by comparing K_{d}, IC₅₀, and Kᵢ's either as measured or calculated depending on the assay; or as a ratio of the same biochemical or biological parameters (e.g., K_{d}, IC₅₀, and Kᵢ's). Such ratios preferably include ratios of VEGFR-1 and VEGFR-3 binding or other measurement to VEGFR-2 binding or other measurement of about 100, about 1,000, about 10,000 or about 100,000.

In certain embodiments, the VEGFR-2 specific inhibitors include those disclosed in U.S. patent application Ser. Nos. 11/482,641 and 11/448,171, and PCT International Application Publication No. WO 05/056764, which are hereby incorporated by reference in its entirety. Sequences may also be further modified, such as by addition of a cysteine in the next to last amino acid of the sequence.

In some embodiments, VEGFR-2 specific inhibitors useful in the methods of the invention comprise a first protein that binds human VEGFR-2 with a binding affinity of about 10 nM or less and binds VEGFR-1 and VEGFR-3 with a binding affinity of about 1 μM or greater; wherein said first protein is substantially a single domain that has substantially monovalent binding with respect to human VEGFR-2, and is substantially free of microbial contamination making it suitable for in vivo administration. The VEGFR-2 specific inhibitors may further comprise a second protein linked by a peptide bond to said first protein, wherein said second protein binds human VEGFR-2 with a binding affinity of about 10 nM or less and binds VEGFR-1 and VEGFR-3 with a binding affinity of about 1 μM or greater. The VEGFR-2 specific inhibitors may further comprise a second linked by at least one peptide bond to said first protein (including a linkage through another protein domain), wherein said second protein binds human serum albumin with a binding affinity of about 300 nM or less. The VEGFR-2 specific inhibitors of matter preferably includes a first protein that binds human VEGFR-2 with an binding affinity of about 100 pM or less and binds VEGFR-1 and VEGFR-3 with a binding affinity of about 1 μM or greater. The VEGFR-2 specific inhibitors more preferably includes a first protein binds human VEGFR-2 with an binding affinity of about 10 pM or less and binds VEGFR-1 and VEGFR-3 with a binding affinity of about 1 μM or greater. Such first protein is preferably a fibronectin based scaffold, such as an Adnectin™, which is an example of a monospecific protein with monovalent binding to VEGFR-2, and may include other operably linked proteins. The VEGFR-2 specific inhibitors may further comprise a second protein linked by PEG to said first protein, wherein said second protein binds human VEGFR-2 with a binding affinity of about 10 nM or less and binds VEGFR-1 and VEGFR-3 with a binding affinity of about 1 μM or greater. Preferably the first and second proteins are fibronectin based scaffolds, such as Adnectins™, which is an example of mono-specific protein with bivalent binding to VEGFR-2. Preferably, PEG is between about 5 kD and about 50 kD.

An aspect of the methods of the invention include VEGFR-2 specific inhibitors, comprising a first protein that specifically binds human VEGFR-2; wherein said first protein is between about 4 kD and about 40 kD in molecular weight, has less than about 30 percent amino acid identity to human VEGF-A, VEGF-C, or VEGF-D and is substantially free of microbial contamination making it suitable for in vivo administration. The VEGFR-2 specific inhibitors can further comprise a PK modulation moiety as described herein. The VEGFR-2 specific inhibitors may have a PK modulation moiety that comprises a PEG moiety covalently linked to said first protein. Such a linkage is not limited to a Cys or Lys amino acid, but there are preferred, particularly with a fibronectin based scaffold, such as an Adnectin™ as a protein that binds VEGFR-2. The VEGFR-2 specific inhibitors may have a PK modulation moiety that comprises a second protein that binds a human protein that increases the half life of said first protein and is operably linked to said first protein. Such a human protein may be human serum albumin. The VEGFR-2 specific inhibitors may appropriately include a PK modulation moiety that comprises a second protein that binds human serum albumin, said second protein is covalently linked to said first protein, and said first protein has a disassociation constant of about 1 nM or less for human VEGFR-2. The VEGFR-2 specific inhibitors may include said first protein that has a disassociation constant of about 10 pM or less for human VEGFR-2. The VEGFR-2 specific inhibitors preferably induces apoptosis in a cell based assay in a cell line dependent on VEGFR-2 activation. The VEGFR-2 specific inhibitors may be designed to have the property that said composition of matter blocks the binding of one or more ligands such as VEGF-A, VEGF-C, or VEGF-D to VEGFR-2. The VEGFR-2 specific inhibitors preferably blocks the binding of VEGF-A, VEGF-C, and/or VEGF-D to VEGFR-2 and further comprises a PEG of at least about 10 kD covalently attached to said first protein.

PEG may be used in many aspects of the invention and different sizes may be used as described herein for the desired therapeutic or other in vivo effect, such as imaging. Larger PEGs are preferred to increase half life in the body, blood, non-blood extracellular fluids or tissues. For in vivo cellular activity, PEGs of the range of about 10 to 60 kD are preferred, as well as PEGs less than about 100 kD and more preferably less than about 60 kD, thought sizes greater than about 100 kD can be used as well.

Preferably proteins useful in methods of the invention are comprised of a fibronectin based scaffold, such as an Adnectin™. The first or second protein or both can be a fibronectin based scaffold, such as an Adnectin™ and can be used in the embodiments described herein. The can include monospecific, bispecific, trispecific and other multi-specific forms of the invention. This includes aspects of the invention that comprise PEG and aspects of the invention that comprises polypeptide linkers or other linkers that are not PEG based. Generally, Adnectins™ are derivatives of fibronectin, particularly the 10^{th} domain.

Also useful in methods of the invention, are PEG based composition of matters, comprising a first PEG linked to a first protein that binds human VEGFR-2 with a binding affinity of about 100 nM and a second protein that binds a human protein; wherein said PEG is between about 5 kD and about 100 kD, and said composition of matter is substantially free of PEG and is substantially free of microbial contamination making it suitable for in vivo administration. Preferably, PEG is linked to said first protein via a Cys and said second protein via a Cys. Alternatively it may be desired to have a PEG linked to said first protein via a Cys and said second protein via a Lys. Heterofunctional PEG can be used to manufacture such embodiments and other embodiments described herein. The PEG based composition of matter can include a second protein that binds human serum albumin with a binding affinity of about 300 nM or less. The PEG based composition of matter may include said first protein that binds human VEGFR-2 with an binding affinity of about 100 pM or less and binds an unrelated receptor, such as human insulin receptor, VEGFR-1 and VEGFR-3 with a binding affinity of about 1 μM or greater. Preferably said second protein binds at least one human tyrosine receptor with an binding affinity of about 1 nM or less and binds an unrelated receptor, such as human insulin receptor, with a binding affinity of about 1 μM or greater.

The PEG based composition of matter may include a protein of the invention that binds at least one of the human versions of the following protein ligands: Ang1, Ang2, FGF (fibroblast growth factor), EGF (epidermal growth factor), HGF (hepatocyte growth factor), VEGF-A (vascular endothelia growth factor-A), VEGF-C, and VEGF-D. Such proteins of the invention may be used with other proteins of the invention to create monospecific or multispecific proteins that can be used in the treatment methods of the invention. Preferably, such proteins are linked by PEG.

The PEG based composition of matter preferably includes molecules wherein said first protein is a fibronectin based scaffold, such as an Adnectin™ and said second protein is a fibronectin based scaffold, such as an Adnectin™. The PEG based composition of matter may alternatively include a first or second protein or both a single chain antibody moiety.

Protein based compositions of matter useful in methods of the invention, comprise a first protein that binds human VEGFR-2 operably linked to a second protein that binds a human protein; wherein said first protein binds human VEGFR-2 with a binding affinity of about 10 nM or less and binds human VEGFR-1 and VEGFR-3 with a binding affinity of about 1 μM or greater, and is substantially free of microbial contamination making it suitable for in vivo administration. Preferably, said first protein and said second protein collectively have 1 or 0 disulfide bonds. This may be desirable to improve protein production in cell based systems. Preferably, said first protein is substantially a single domain that has substantially monovalent binding to VEGFR-2. This may be desirable to improve protein production in cell based systems. Preferably, said first protein binds human VEGFR-2 with an binding affinity of about 100 pM or less and has at least two structural loops that participate in the binding of said first protein to human VEGFR-2. Preferably, said second protein is a fibronectin based scaffold, such as an Adnectin™ linked by at least one peptide bond to said first protein. In some embodiments, said first protein and second protein are linked by at least one disulfide bond. Though this can be accomplished in cells in may be desired to perform this linkage in vitro without cells. The protein based composition of matter may include a first protein that binds human VEGFR-2 with an binding affinity of about 300 pM or less and has at least two structural loops that participate in the binding of said first protein to human VEGFR-2. Preferably, said first protein binds human VEGFR-2 with an binding affinity of about 1 nM or less and binds human VEGFR-1 and VEGFR-3 with a binding affinity of about 1 μM or greater. Preferably, the protein based composition of matter further comprises a PEG moiety operably linked either said first protein or said second protein. Preferably, said second protein binds a human tyrosine kinase receptor up-regulated in a human cancer, wherein said second protein binds the human tyrosine kinase receptor with an binding affinity of about 10 nM or less and binds an unrelated receptor, such as human insulin receptor, with a binding affinity of about 1 μM or greater.

Protein therapeutics useful in methods of the invention comprise a first protein moiety that binds human VEGFR-2 operably linked to a second protein moiety that binds a human therapeutic target; wherein said first protein binds human VEGFR-2 with a binding affinity of about 10 nM or less and binds human VEGFR-1 and VEGFR-3 with a binding affinity of about 1 μM or greater, and is substantially free of microbial contamination making it suitable for in vivo administration; and further wherein said second protein binds the human therapeutic target with a binding affinity of about 10 nM or less and binds an unrelated receptor, such as human insulin receptor, with a binding affinity of about 1 μM or greater, and is substantially free of microbial contamination making it suitable for in vivo administration. The protein therapeutic may include a first protein moiety and said second protein moiety collectively have at least 6 disulfide bonds. Preferably, said first protein and said second protein are a single polypeptide expressed from a microbe. Preferably, said first protein binds human VEGFR-2 with a binding affinity of about 100 pM or less and has at least two structural loops that participate in the binding of said first protein to human VEGFR-2. The protein therapeutic includes an embodiment wherein at least one of said first protein moiety and said second protein moiety is an antibody moiety. Preferably, such antibody moiety is less than about 50 kD. The protein therapeutic includes an embodiment wherein at least one of said first protein moiety and said second protein moiety is a single chain antibody moiety. The protein therapeutic includes an embodiment wherein said second protein moiety binds one of the human versions of the following proteins: receptors: EGFR, folate receptor, Her2, Her3, c-kit, c-Met, FGFRI, FGFR2, PDGFR, VEGFR1, VEGFR2, VEGFR3, and Tie2; and ligands: Ang1, Ang2, FGF (fibroblast growth factor), EGF (epidermal growth factor), HGF (hepatocyte growth factor), stem cell factor (SCF), VEGF-A (vascular endothelia growth factor-A), VEGF-C, and VEGF-D. The protein therapeutic includes an embodiment wherein at least one of said first protein moiety and said second protein moiety is a derivative of lipocalin. The protein therapeutic includes an embodiment wherein at least one of said first protein moiety and said second protein moiety is a derivative of a tetranectin. The protein therapeutic includes an embodiment wherein at least one of said first protein moiety and said second protein moiety is a derivative of an avimer.

Protein therapeutics useful in methods of the invention comprise a first protein moiety that binds human VEGFR-2 operably linked to a PEG that is operable linked to a second protein moiety that binds a human therapeutic target; wherein said first protein moiety binds human VEGFR-2 with a binding affinity of about 10 nM or less and binds human VEGFR-1 and VEGFR-3 with a binding affinity of about 1 μM or greater, and is substantially free of microbial contamination making it suitable for in vivo administration; and further wherein said second protein moiety binds the human therapeutic target with a binding affinity of about 10 nM or less and binds an unrelated receptor, such as human insulin receptor, with a binding affinity of about 1 μM or greater, and is substantially free of microbial contamination making it suitable for in vivo administration. The protein therapeutic includes an embodiment wherein said first protein moiety and said second protein moiety collectively have at least about 8 disulfide bonds. The protein therapeutic includes an embodiment wherein said first protein and said second protein are a single polypeptide expressed from a microbe.

The protein therapeutic includes an embodiment wherein said first protein binds human VEGFR-2 with an binding affinity of about 100 pM or less and has at least two structural loops that participate in the binding of said first protein to human VEGFR-2. The protein therapeutic includes an embodiment wherein at least one of said first protein moiety and said second protein moiety is an antibody moiety of less than about 40 kD. The protein therapeutic includes an embodiment wherein at least one of said first protein moiety and said second protein moiety is a single chain antibody moiety. The protein therapeutic includes embodiments wherein at least one of said first protein moiety and said second protein moiety is a derivative of lipocalin, a tetranectin, an avimer, and a ankyrin.

Protein therapeutics useful in methods of the invention, comprise a first protein moiety that binds human VEGFR-2 operably linked to a PEG that is operable linked to a second protein moiety that binds a human therapeutic target; wherein said first protein moiety binds human VEGFR-2 with a binding affinity of about 10 nM or less and binds human VEGFR-1 and VEGFR-3 with a binding affinity of about 1 μM or greater and binding with respect to human VEGFR-2 is substantially monovalent, and is substantially free of microbial contamination making it suitable for in vivo administration; and further wherein said second protein moiety binds the human therapeutic target with a binding affinity of about 10 nM or less and binds an unrelated receptor, such as human insulin receptor, with a binding affinity of about 1 μM or greater and is substantially free of microbial contamination making it suitable for in vivo administration. Substantially monovalent binding to VEGFR-2 is helpful in reducing VEGFR-2 activation in this and other embodiments of the invention. In some instances, VEGFR-2 may be caused by proteins such as full length antibodies or Fabs that have multivalent binding to VEGFR-2, e.g., avidity. In addition, Fabs and full length antibodies have more rigid conformations that may mimic activating ligands. This may also cause receptor dimerization. The protein therapeutic includes an embodiment wherein said PEG is at least about 5 kD, at least about 10 kD, at least about 20 kD, at least about 40 kD, and at least about 50 kD. The protein therapeutic includes an embodiment wherein said first protein is operably linked to PEG through a single Cys or Lys. Preferably, said first protein has no more than a single Cys in this and other embodiments. The protein therapeutic includes an embodiment wherein said second protein is operably linked to PEG through a single Cys or Lys. Preferably, said second protein has no more than a single Cys in this and other embodiments. The protein therapeutic includes an embodiment wherein said single Cys or Lys is located in said first protein in a non-wildtype location in the amino acid sequence. The protein therapeutic includes an embodiment wherein said single Cys or Lys is located in said second protein in a non-wildtype location in the amino acid sequence.

Protein therapeutics useful in methods of the invention, comprise a first protein moiety that binds human VEGFR-2 operably linked to a biocompatible polymer that is operable linked to a second protein moiety that binds a human therapeutic target; wherein said first protein moiety binds human VEGFR-2 with a binding affinity of about 10 nM or less and binds an unrelated receptor, such as human insulin receptor, with a binding affinity of about 1 μM or greater, and is substantially free of microbial contamination making it suitable for in vivo administration; further wherein said second protein moiety binds the human therapeutic target with a binding affinity of about 10 nM or less and binds an unrelated receptor, such as human insulin receptor, with a binding affinity of about 1 μM or greater, and is substantially free of microbial contamination making it suitable for in vivo administration; and further wherein said first protein moiety and second protein moiety have affinities for their respective targets that are optimized to minimize non-therapeutic affects and to maximize therapeutic benefit of binding to more than one therapeutic target.

The protein therapeutic includes an embodiment with a biocompatible polymer linker comprises a polypeptide. The protein therapeutic includes an embodiment wherein said biocompatible polymer is a PEG. The protein therapeutic includes an embodiment wherein said PEG is at least about 30 kD. The protein therapeutic includes an embodiment wherein said first protein moiety induces apoptosis. The protein therapeutic includes an embodiment wherein said first protein moiety inhibits cell proliferation, blocks VEGF binding and does not activate human VEGFR-2 at sub IC₅₀ concentrations in a cell based assay. The protein therapeutic may further comprise a pharmaceutically acceptable formulation for IV, IP or subcutaneous administration in this and other embodiments.

The protein therapeutic may include PEG that is at least about 20 kD or about 30 kD. PEG along with the first protein moiety may facilitate induction of apoptosis. Larger PEGs are preferred for this purpose. PEG embodiments with monospecific activity and multivalent (e.g., bivalent) binding to VEGFR-2 are particularly preferred to block VEGFR-2 activities such as control of apoptosis, phosphorylation or dimerization. Preferably, said first protein moiety inhibits cell proliferation, blocks VEGF-A, VEGF-C, or/and VEGF-D binding and does not activate human VEGFR-2 at sub IC₅₀ concentrations in a cell based assay. The protein therapeutic may further comprise a pharmaceutically acceptable formulation for IV, IP or subcutaneous administration. Preferably, said first protein moiety induces apoptosis in cell based assays with an IC₅₀ of less than about 1 nM or about 10 pM. Preferably, first protein moiety inhibits cell proliferation, blocks VEGF-A, VEGF-C, and/or VEGF-D binding and does not activate human VEGFR-2 at sub IC₅₀ concentrations in a cell based assay with an IC₅₀ of less than about 1 nM or about 100 pM.

Proteins useful in methods of the invention include embodiments wherein a first or second protein or polypeptide linker has a protease site that is cleavable by a protease in the blood or target tissue. Such embodiments can be used to release two or more therapeutic proteins for better delivery or therapeutic properties or more efficient production compared to separately producing such proteins.

Proteins useful in methods of the invention include embodiments wherein two or more proteins or polypeptide linker using a biocompatible polymer such as a polymeric sugar. Such polymeric sugar can include an enzymatic cleavage site that is cleavable by an enzyme in the blood or target tissue. Such embodiments can be used to release two or more therapeutic proteins for better delivery or therapeutic properties or more efficient production compared to separately producing such proteins.

Methods of the invention includes a protein therapeutic, comprising a first protein that binds human VEGFR-2 operably linked to a PEG that is operable linked to a second protein that binds a human protein; wherein said first protein binds human VEGFR-2 with a binding affinity of about 10 nM or less and binds human VEGFR-1 and VEGFR-3 with a binding affinity of about 1 μM or greater and is an antagonist of human VEGFR-2, and is substantially free of microbial contamination making it suitable for in vivo administration; and further wherein said second protein binds the human therapeutic target with a binding affinity of about 10 nM or less and binds an unrelated target, such as human insulin receptor, with a binding affinity of about 1 μM or greater, and is substantially free of microbial contamination making it suitable for in vivo administration.

The protein therapeutic includes an embodiment wherein said first protein inhibits cell proliferation, has a Tₘ of at least 55° C., and non-wildtype Cys or Lys in region of its amino acid sequence that does not substantially interfere with binding to human VEGFR-2. The protein therapeutic includes an embodiment wherein said first protein and said second protein are a single polypeptide expressed from a microbe. The protein therapeutic includes an embodiment wherein said first protein binds human VEGFR-2 with a binding affinity of about 50 pM or less and has at least two structural loops that participate in the binding of said first protein to human VEGFR-2. The protein therapeutic includes an embodiment wherein said protein therapeutic has a half life in in vivo of at least one day with IV administration. The protein therapeutic includes an embodiment wherein said protein therapeutic has an exposure level at a concentration of at least about 10 times the binding affinity of said first protein to human VEGFR-2 for over 24 hours in a rodent after administration. The protein therapeutic includes an embodiment wherein said protein therapeutic has an exposure level at a concentration of at least about 100 times the binding affinity of said first protein to human VEGFR-2 for over 24 hours in a rodent after subcutaneous administration. The protein therapeutic includes an embodiment wherein said first protein binds human VEGFR-2 with a binding affinity of about 100 pM or less and has at least two structural loops that participate in the binding of said first protein to human VEGFR-2. Preferably, said first or second protein or both is a fibronectin based scaffold, such as an Adnectin™.

Additionally, tumor-associated targets may be targeted in methods of the invention. In some embodiments antigen targeting will help localize the therapeutic in terms of tissue distribution or increased local concentration affect either in the tissue or desired cell type. Alternatively, it may provide an additional mechanism of action to combat cancer along with one of the targets described herein for which a therapeutic is made. Such antigens or targets include, but are not limited to, carbonic anhydrase IX, A3, antigen specific for A33 antibody, BrE3-antigen, CD1, CD1a, CD3, CD5, CD15, CD16, CD19, CD20, CD21, CD22, CD23, CD25, CD30, CD45, CD74, CD79a, CD80, HLA-DR, NCA 95, NCA90, HCG and its subunits, CEA (CEACAM-5), CEACAM-6, CSAp, EGFR, EGP-1, EGP-2, Ep-CAM, Ba 733, HER2/neu, hypoxia inducible factor (HIF), KC4-antigen, KS-1-antigen, KS1-4, Le-Y, macrophage inhibition factor (MIF), MAGE, MUC1, MUC2, MUC3, MUC4, PAM-4-antigen, PSA, PSMA, RS5, S100, TAG-72, p53, tenascin, IL-6, IL-8, insulin growth factor-I (IGF-I), insulin growth factor-II (IGF-II), Tn antigen, Thomson-Friedenreich antigens, tumor necrosis antigens, placenta growth factor (P1GF), 17-1A-antigen, an angiogenesis marker (e.g., ED-B fibronectin), an oncogene marker, an oncogene product, and other tumor-associated antigens. Recent reports on tumor associated antigens include Mizukami et al. (2005, Nature Med. 11:992-97); Hatfield et al. (2005, Curr. Cancer Drug Targets 5:229-48); Vallbohmer et al. (2005, J. Clin. Oncol. 23:3536-44); and Ren et al. (2005, Ann. Surg. 242:55-63), each incorporated herein by reference.

In other embodiments, an anti-angiogenic agent may form a portion of a therapeutic and may be operably linked to a VEGFR-2 specific inhibitor. Exemplary anti-angiogenic agents of use include angiostatin, baculostatin, canstatin, maspin, anti-VEGF antibodies or peptides, anti-placental growth factor antibodies or peptides, anti-Flk-1 antibodies, anti-Flt-1 antibodies or peptides, laminin peptides, fibronectin peptides, plasminogen activator inhibitors, tissue metalloproteinase inhibitors, interferons, interleukin 12, IP-10, Gro-.beta., thrombospondin, 2-methoxyoestradiol, proliferin-related protein, carboxiamidotriazole, CM101, Marimastat, pentosan polysulphate, angiopoietin 2, interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

The invention provides kits useful in the treatment of metastatic cancer comprising one or more of the elements described herein, and instructions for the use of those elements. In a preferred embodiment, a kit of the present invention includes a protein of the invention, alone or with a second a therapeutic agent. The instructions for this preferred embodiment include instructions for inhibiting the growth of a cancer cell using a protein of the invention, alone or with a second therapeutic agent, and/or instructions for a method of treating a patient having a cancer using the same.

### BRIEF DESCRIPTION OF THE DRAWINGS

FIG. 1 shows the effect of Comp-I and bevacizumab in an MDA-MB-231 orthotopic lung cancer metastasis model. MDA-MB-231 breast tumor cells implanted in mammary fat pad of mice and resultant tumors resected day 24. Drug treatment was initiated on day 28 and mice were sacrificed at 118 days and local regrowth and lung metastases evaluated.

### DETAILED DESCRIPTION OF THE INVENTION

Definitions

“Metastasis”, as used herein, is defined as the migration or transfer of malignant tumor cells, or neoplasms, via the circulatory or lymphatic systems or via natural body cavities, usually from the primary focus of tumor, cancer or a neoplasia to a distant site in the body, and the subsequent development of one or a plurality of secondary tumors or colonies thereof in the one new or the plurality of new locations. “Metastases” includes the secondary tumors or colonies formed as a result of metastasis and encompasses micro-metastases.

As used herein, the terms “treatment”, “treating”, and the like, refer to obtaining a desired pharmacologic and/or physiologic effect. The effect may be prophylactic in terms of completely or partially preventing a disorder or symptom thereof and/or may be therapeutic in terms of a partial or complete cure for a disorder and/or adverse affect attributable to the disorder. “Treatment”, as used herein, covers any treatment of a disease in a mammal, particularly in a human, and includes: (a) increasing-survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease. Specifically, the disease/disorder is metastases or the development thereof.

As used herein, a therapeutic that “prevents” a disorder or condition is a compound that, in a statistical sample, reduces the occurrence of the disorder or condition in the treated sample relative to an untreated control sample, or delays the onset or reduces the severity of one or more symptoms of the disorder or condition relative to the untreated control sample. An agent that prevents metastases may completely block the development of metastases or reduce the number of metastases that form relative to control.

By a “polypeptide” is meant any sequence of two or more amino acids, regardless of length, post-translation modification, or function. “Polypeptide,” “peptide,” and “protein” are used interchangeably herein. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D). The term “single domain polypeptide” is used to indicate that the target binding activity (e.g., VEGFR-2 binding activity) of the subject polypeptide is situated within a single structural domain, as differentiated from, for example, antibodies and single chain antibodies, where antigen binding activity is generally contributed by both a heavy chain variable domain and a light chain variable domain. It is contemplated that a plurality of single domain polypeptides of the sort disclosed herein could be connected to create a composite molecule with increased avidity. Likewise, a single domain polypeptide may be attached (e.g., as a fusion protein) to any number of other polypeptides, such as fluorescent polypeptides, targeting polypeptides and polypeptides having a distinct therapeutic effect.

Single domain polypeptides of either the immunoglobulin or immunoglobulin-like scaffold will tend to share certain structural features. For example, the polypeptide may comprise between about 80 and about 150 amino acids, which amino acids are structurally organized into a set of beta or beta-like strands, forming beta sheets, where the beta or beta-like strands are connected by intervening loop portions. The beta sheets form the stable core of the single domain polypeptides, while creating two “faces” composed of the loops that connect the beta or beta-like strands. As described herein, these loops can be varied to create customized ligand binding sites, and, with proper control, such variations can be generated without disrupting the overall stability of the protein. In antibodies, three of these loops are the well-known Complementarity Determining Regions (or “CDRs”).

Scaffolds for formation of a single domain polypeptides should be highly soluble and stable in physiological conditions. Examples of immunoglobulin scaffolds are the single domain V_{H} or V_{L} scaffold, as well as a single domain camelid V_{HH} domain (a form of variable heavy domain found in camelids) or other immunoglobulin variable domains found in nature or engineered in the laboratory. In the single domain format disclosed herein, an immunoglobulin polypeptide need not form a dimer with a second polypeptide in order to achieve binding activity. Accordingly, any such polypeptides that naturally contain a cysteine which mediates disulfide cross-linking to a second protein can be altered to eliminate the cysteine. Alternatively, the cysteine may be retained for use in conjugating additional moieties, such as PEG, to the single domain polypeptide.

Other scaffolds may be non-antibody scaffold proteins. By “non-antibody scaffold protein or domain” is meant a non-antibody polypeptide having an immunoglobulin-like fold. By “immunoglobulin-like fold” is meant a protein domain of between about 80-150 amino acid residues that includes two layers of antiparallel beta-sheets, and in which the flat, hydrophobic faces of the two beta-sheets are packed against each other. An example of such a scaffold is the “fibronectin-based scaffold protein”, by which is meant a polypeptide based on a fibronectin type III domain (Fn3). Fibronectin is a large protein which plays essential roles in the formation of extracellular matrix and cell-cell interactions; it consists of many repeats of three types (types I, II, and III) of small domains (Baron et al. Philos. Trans. R. Soc. Lond. B. Biol. Sci. 1991 May 29; 332(1263):165-70). Fn3 itself is the paradigm of a large subfamily which includes portions of cell adhesion molecules, cell surface hormone and cytokine receptors, chaperoning, and carbohydrate-binding domains. For reviews see Bork and Doolittle, Proc. Natl. Acad. Sci. USA. 1992 Oct. 1; 89(19):8990-4; Bork et al. J. Mol. Biol. 1994 Sep. 30; 242(4):309-20; Campbell and Spitzfaden, Structure. 1994 May 15; 2(5):333-7; Harpez and Chothia, J. Mol. Biol. 1994 May 13; 238(4):528-39).

Preferably, the fibronectin-based scaffold protein is a “¹⁰FN3” scaffold, by which is meant a polypeptide variant based on the tenth module of the human fibronectin type III protein in which one or more of the solvent accessible loops has been randomized or mutated, particularly one or more of the three loops identified as the BC loop (amino acids 23-30), DE loop (amino acids 52-56) and FG loop (amino acids 77-87) (the numbering scheme is based on the sequence on the tenth Type III domain of human fibronectin, with the amino acids Val-Ser-Asp-Val-Pro (SEQ ID NO:62) representing amino acids numbers 1-5). The amino acid sequence of the wild-type tenth module of the human fibronectin type III domain is: VSDVPRDLEVVAATPTSLLISWDAPAVTVRYYRITYGETGGNSPVQEFTVPG SKSTATISGLKPGVDYTITGYAVTGRGDSPASSKPISINYRT (SEQ ID NO:1). Thus, the wild-type BC loop comprises the sequence of DAPAVTVR (SEQ ID NO:63); the wild-type DE loop comprises the sequence of GSKST (SEQ ID NO:64); the wild-type FG loop comprises the sequence of GRGDSPASSKP (SEQ ID NO:65). The sequences flanking the BC, DE, and FG loops are also termed Frameworks 1, 2, 3, and 4. Preferably, the fibronectin based scaffold is based on SEQ ID NO:1.

A variety of improved mutant ¹⁰Fn3 scaffolds have been identified. A modified Asp7, which is replaced by a non-negatively charged amino acid residue (e.g., Asn, Lys, etc.). Both of these mutations have the effect of promoting greater stability of the mutant ¹⁰Fn3 at neutral pH as compared to the wild-type form. A variety of additional alterations in the ¹⁰Fn3 scaffold that are either beneficial or neutral have been disclosed. See, for example, Batori et al. Protein Eng. 2002 December; 15(12):1015-20; Koide et al. Biochemistry 2001 Aug. 28; 40(34):10326-33.

Both the variant and wild-type ¹⁰Fn3 proteins are characterized by the same structure, namely seven beta-strand domain sequences (designated A through and six loop regions (AB loop, BC loop, CD loop, DE loop, EF loop, and FG loop) which connect the seven beta-strand domain sequences. The beta strands positioned closest to the N- and C-termini may adopt a beta-like conformation in solution. In SEQ ID NO:1, the AB loop corresponds to residues 15-16, the BC loop corresponds to residues 22-30, the CD loop corresponds to residues 39-45, the DE loop corresponds to residues 51-55, the EF loop corresponds to residues 60-66, and the FG loop corresponds to residues 76-87. The BC loop, DE loop, and FG loop are all located at the same end of the polypeptide. Similarly, immunoglobulin scaffolds tend to have at least seven beta or beta-like strands, and often nine beta or beta-like strands. Adnectins™ can include other Fn3 type fibronectin domains as long as they exhibit useful activities and properties of ¹⁰Fn3 type domains.

A single domain polypeptide disclosed herein may have at least five to seven beta or beta-like strands distributed between at least two beta sheets, and at least one loop portion connecting two beta or beta-like strands, which loop portion participates in binding to VEGFR-2, with the binding characterized by a dissociation constant that is less than 1×10⁻⁶ M, and preferably less than 1×10⁻⁸ M. As described herein, polypeptides having a dissociation constant of less than 5×10⁻⁹ M are particularly desirable for therapeutic use in vivo to inhibit VEGF signaling. Polypeptides having a dissociation constant of between 1×10⁻⁶ M and 5×10⁻⁹ M may be desirable for use in detecting or labeling, ex vivo or in vivo, VEGFR-2 proteins.

Optionally, the “VEGFR-2 binding protein” will bind specifically to VEGFR-2 relative to other related proteins from the same species. By “specifically binds” is meant a polypeptide that recognizes and interacts with a target protein (e.g., VEGFR-2) but that does not substantially recognize and interact with other molecules in a sample, for example, a biological sample. In preferred embodiments a polypeptide of the invention will specifically bind a VEGFR-2 with a K_{d} at least as tight as 500 nM. Preferably, the polypeptide will specifically bind a VEGFR-2 with a K_{d} of 1 pM to 500 nM, more preferably 1 pM to 100 nM, more preferably 1 pM to 10 nM, and most preferably 1 pM to 1 nM or lower.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays known in the art for evaluating such antibody effector functions.

A “native sequence Fc region” comprises an amino acid sequence identical to the amino acid sequence of an Fc region found in nature.

A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Natl. Acad. Sci. USA 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g., from blood or PBMCs as described herein.

The terms “Fc receptor” and “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daeron, Annu. Rev. Immunol. 15:203-234 (1997)). FcRs are reviewed in Ravetch and Kinet, Annu. Rev. Immunol. 9:457-92 (1991); Capel et al. Immunomethods 4:25-34 (1994); and de Haas et al. J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al. J. Immunol. 117:587 (1976); and Kim et al. J. Immunol. 24:249 (1994)).

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

A “polypeptide chain” is a polypeptide wherein each of the domains thereof is joined to other domain(s) by peptide bond(s), as opposed to non-covalent interactions or disulfide bonds.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In preferred embodiments, the polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

Targets may also be fragments of said targets. Thus a target is also a fragment of said target, capable of eliciting an immune response. A target is also a fragment of said target, capable of binding to a single domain antibody raised against the full length target.

A fragment as used herein refers to less than 100% of the sequence (e.g., 99%, 90%, 80%, 70%, 60%, 50%, 40%, 30%, 20%, 10% etc.), but comprising 5, 6, 7, 8, 9, 10, 12, 13, 14, 15, 16, 17, 18, 19, 20, 21, 22, 23, 24, 25 or more amino acids. A fragment is of sufficient length such that the interaction of interest is maintained with affinity of 1×10⁻⁶ M or better.

A fragment as used herein also refers to optional insertions, deletions and substitutions of one or more amino acids which do not substantially alter the ability of the target to bind to a single domain antibody raised against the wild-type target. The number of amino acid insertions deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

A protein of the invention that “induces cell death” is one which causes a viable cell to become nonviable. The cell is generally one which expresses the antigen to that the protein binds, especially where the cell overexpresses the antigen. Preferably, the cell is a cancer cell, e.g., a breast, ovarian, stomach, endometrial, salivary gland, lung, kidney, colon, thyroid, pancreatic or bladder cell. In vitro, the cell may be a SKBR3, BT474, Calu 3, MDA-MB453, MDA-MB-361 or SKOV3 cell. Cell death in vitro may be determined in the absence of complement and immune effector cells to distinguish cell death induced by antibody dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the protein of the invention is able to induce cell death, loss of membrane integrity as evaluated by uptake of propidium iodide (PI), trypan blue (see Moore et al. Cytotechnology 17:1-11 (1995)) or 7AAD can be assessed relative to untreated cells.

A protein of the invention that “induces apoptosis” is one that induces programmed cell death as determined by binding of apoptosis related molecules or events, such as annexin V, fragmentation of DNA, cell shrinkage, dilation of endoplasmic reticulum, cell fragmentation, and/or formation of membrane vesicles (called apoptotic bodies). The cell is one which expresses the antigen to which the protein binds and may be one which overexpresses the antigen. The cell may be a tumor cell, e.g., a breast, ovarian, stomach, endometrial, salivary gland, lung, kidney, colon, thyroid, pancreatic or bladder cell. In vitro, the cell may be a SKBR3, BT474, Calu 3 cell, MDA-MB453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering as disclosed in the example herein; and nuclear/chromatin condensation along with DNA fragmentation can be evaluated by any increase in hypodiploid cells. Preferably, the protein that induces apoptosis is one which results in about 2- to 50-fold, preferably about 5 to 50-fold, and most preferably about 10 to 50-fold, induction of annexin binding relative to untreated cell in an annexin binding assay using cells expressing the antigen to which the protein of the invention binds.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the time to disease progression (TTP) and/or determining the response rates (RR).

The term “PK” is an acronym for “pharmokinetic” and encompasses properties of a compound including, by way of example, absorption, distribution, metabolism, and elimination by a subject. A “PK modulation protein” refers to any protein or peptide that affects the pharmokinetic properties of a biologically active molecule when fused to or administered together with the biologically active molecule. Examples of a PK modulation protein include PEG, as well as human serum albumin (HSA) binders as disclosed in U.S. patent application Ser. Nos. 11/106,415 and 11/331,415.

Overview

The present application provides novel methods of treating, preventing, or reducing the severity of metastases. The application results, in part, on the surprising discovery that selective inhibition of VEGFR2 reduces the prevalence of metastases in a cancer model.

The methodology described herein has been successfully used to develop proteins of the invention that will be useful in the treatment of metastatic cancer, that include but are not limited to, single domain VEGFR-2 binding polypeptides derived from at least two related groups of protein structures: those proteins having an immunoglobulin fold and those proteins having an immunoglobulin-like fold.

Treatment for Metastases

In one aspect, the application provides methods for treating patients afflicted with metastatic cancer, comprising the administration of a VEGFR-2 specific inhibitor. Administration of the inhibitor results in statistically significant and clinically meaningful improvement of the treated patient as measured by the duration of survival, progression free survival, response rate or duration of response. In some embodiments, the patient is afflicted with metastatic breast, metastatic colorectal, or metastatic lung cancer.

In some embodiments, the patients treated are at risk of developing metastatic cancer. For example, cancer patients may present a high risk of metastasis, depending on the type of cancer. Administration of the inhibitor reduces the risk of developing metastases, reduces the number of metastases, or reduces the size of metastases.

The cancer amendable for treatment by the present invention includes, but not limited to, carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, melanoma, ovarian cancer, mesothelioma, and multiple myeloma. The method of the present invention is particularly suitable for the treatment of vascularized tumors.

In some embodiments, administration of a VEGFR-2 specific inhibitor inhibits metastasis by at least about 10% 20%, 30%, 40%, 60%, 70%, 80%, 90%, or 100%. In some embodiments, methods of inhibiting metastasis to lymph node is provided. In some embodiments, methods of inhibiting metastasis to the lung is provided.

Combination Chemotherapy

In other therapeutic treatments, VEGFR-2 inhibitors are co-administered, or administered sequentially, with one or more additional therapeutic agents. Suitable therapeutic agents include, but are not limited to, targeted therapeutics, other targeted biologics, and cytotoxic or cytostatic agents. In some instances in will be preferred to administer agents from the same or separate therapeutically acceptable vial, syringe or other administration device that holds a liquid formulation.

Cancer therapeutic agents are those agents that seek to kill or limit the growth of cancer cells while having minimal effects on the patient. Thus, such agents may exploit any difference in cancer cell properties (e.g., metabolism, vascularization or cell-surface antigen presentation) from healthy host cells. Differences in tumor morphology are potential sites for intervention: for example, the second therapeutic can be an antibody such as an anti-VEGF antibody that is useful in retarding the vascularization of the interior of a solid tumor, thereby slowing its growth rate. Other therapeutic agents include, but are not limited to, adjuncts such as granisetron HCl, androgen inhibitors such as leuprolide acetate, antibiotics such as doxorubicin, antiestrogens such as tamoxifen, antimetabolites such as interferon alpha-2a, cytotoxic agents such as taxol, enzyme inhibitors such as ras farnesyl-transferase inhibitor, immunomodulators such as aldesleukin, and nitrogen mustard derivatives such as melphalan HCl, and the like.

In some embodiments, the present invention provides a method for increasing the duration of survival of a human patient having or at risk of developing metastatic cancer, comprising administering to the patient effective amounts of a VEGFR-2 specific inhibitor and an anti-neoplastic composition, wherein said anti-neoplastic composition comprises at least one chemotherapeutic agent, whereby the co-administration of the VEGFR-2 specific inhibitor and the anti-neoplastic composition effectively increases the duration of survival.

In some embodiments, the present invention provides a method for increasing the progression free survival of a human patient having or at risk of developing metastatic cancer, comprising administering to the patient effective amounts of a VEGFR-2 specific inhibitor and an anti-neoplastic composition, wherein said anti-neoplastic composition comprises at least one chemotherapeutic agent, whereby the co-administration of the VEGFR-2 specific inhibitor and the anti-neoplastic composition effectively increases the duration of progression free survival.

Furthermore, the present invention provides a method for treating a group of human patients having or at risk of developing metastatic cancer, comprising administering to the patient effective amounts of a VEGFR-2 specific inhibitor and an anti-neoplastic composition, wherein said anti-neoplastic composition comprises at least one chemotherapeutic agent, whereby the co-administration of the VEGFR-2 specific inhibitor and the anti-neoplastic composition effectively increases the response rate in the group of patients.

In yet another aspect, the present invention provides a method for increasing the duration of response of a human patient having or at risk of developing metastatic cancer, comprising administering to the patient effective amounts of a VEGFR-2 specific inhibitor and an anti-neoplastic composition, wherein said anti-neoplastic composition comprises at least one chemotherapeutic agent, whereby the co-administration of the VEGFR-2 specific inhibitor and the anti-neoplastic composition effectively increases the duration of response.

Chemotherapeutic agents useful in methods of the invention include alkylating agents such as thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin; carminomycin, carzinophilin, chromomycinis, dactinomycin, daunorubicin, detorubicin, 6-diazo-5-oxo-L-norleucine, ADRIAMYCIN™ doxorubicin (including inorpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

Additional agents include abarelix, altretamine, aminoglutethimide, amsacrine, anastrozole, antide, asparaginase, AZD2171 (Recentin™), Bacillus Calmette-Guerin/BCG (TheraCys™, TICE™), bevacizumab (see U.S. Pat. No. 6,054,297; bevacizumab™), bicalutamide, bleomycin, bortezomib (Velcade™), buserelin, busulfan, campothecin, capecitabine, carboplatin, carmustine, cetuximab (Erbitux™), chlorambucil, cisplatin, cladribine, clodronate, colchicine, cyclophosphamide, cyproterone, cytarabine, dacarbazine, dactinomycin, dasatinib ((see U.S. Pat. No. 6,596,746; Sprycel™), daunorubicin, dienestrol, diethylstilbestrol, dexamethasone, docetaxel (Taxotere™), doxorubicin, Abx-EGF, epothilones, epirubicin, erlonitib (Tarceva™), estradiol, estramustine, etoposide, exemestane, 5-fluorouracil, filgrastim, fludarabine, fludrocortisone, fluorouracil, fluoxymesterone, flutamide, fulvestrant, gefitinib (Iressa™), gemcitabine (see U.S. Pat. No. 4,808,614; Gemzar™), genistein, goserelin, hydroxyurea, idarubicin, ifosfamide, imatinib mesylate (see U.S. Pat. No. 5,521,184; Gleevac™), interferon, irinotecan, ibritumomab (Zevalin™), ironotecan, ixabepilone (BMS-247550), lapatinib (see U.S. Pat. No. 6,391,874; Tykreb™), letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, motesanib diphosphate (AMG 706) nilutamide, nocodazole, octreotide, oxaliplatin, paclitaxel (Taxol™), pamidronate, pentostatin, plicamycin, porfimer, procarbazine, raltitrexed, rapamycin, rituximab (Rituxan™), sorafenib (Nexavar™/Bayer BAY43-9006), streptozocin, suramin, sunitinib malate (see U.S. Pat. No. 6,573,293; Sutent™), tamoxifen, temsirolimus (see U.S. Pat. No. 5,362,718; CCl-779), temozolomide (see U.S. Pat. No. 5,260,291; Temodar™), teniposide, testosterone, thioguanine, thiotepa, titanocene dichloride, topotecan, toremifene, tositumomab (Bexxar™), trastuzumab (U.S. Pat. No. 5,821,337; Herceptin™), tretinoin, VEGF Trap (aflibercept; preparation described in U.S. Pat. No. 5,844,099), vinblastine, vincristine, vindesine, and vinorelbine, zoledronate.

In some embodiments the anti-neoplastic composition is a fluorouracil based combination regimen. The combination regimen may comprise, for example, 5-FU+leucovorin, 5-FU+leucovorin+irinotecan (IFL), or 5-FU+leucorvin+oxaliplatin (FOLFOX).

Also included in this definition of chemotherapeutic agents are anti-hormonal agents that act to regulate or inhibit hormone action on tumors such as anti-estrogens and selective estrogen receptor modulators (SERMs), including, for example, tamoxifen (including NOLVADEX™ tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE™ megestrol acetate, AROMASIN™ exemestane, formestanie, fadrozole, RIVISOR™ vorozole, FEMARA™ letrozole, and ARIMIDEX™ anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME™ ribozyme) and a HER2 expression inhibitor; vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; and pharmaceutically acceptable salts, acids or derivatives of any of the above.

A “growth inhibitory agent” when used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL™, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); epidermal growth factor; hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al. “Prodrugs: A Chemical Approach to Targeted Drug Delivery,” Directed Drug Delivery, Borchardt et al. (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenyl acetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

The one or more additional therapeutic agents can be administered before, concurrently, or after the VEGFR-2 specific inhibitor. The skilled artisan will understand that for each therapeutic agent there may be advantages to a particular order of administration. Similarly, the skilled artisan will understand that for each therapeutic agent, the length of time between which the agent, and the VEGFR-2 specific inhibitor is administered, will vary.

Combination Therapy with Radiation Therapy and Surgery

In another aspect, the present invention provides a method of treating a patient having or at the risk of developing metastatic cancer comprising a first therapy comprising administering a VEGFR-2 specific inhibitor and a second therapy comprising radiation and/or surgery.

The administration of the VEGFR-2 specific inhibitor may be prior to the radiation and/or surgery, after the radiation and/or surgery, or concurrent with the radiation and/or surgery. For example, the administration of the VEGFR-2 specific inhibitor may precede or follow the radiation and/or surgery therapy by intervals ranging from minutes to weeks. In some embodiments, the time period between the first and the second therapy is such that the VEGFR-2 specific inhibitor and the radiation/surgery would still be able to exert an advantageously combined effect on the cell. For example, the VEGFR-2 specific inhibitor may be administered less than about any of 1, 3, 6, 9, 12, 18, 24, 48, 60, 72, 84, 96, 108, 120 hours prior to the radiation and/or surgery. In some embodiments, the VEGFR-2 specific inhibitor is administered less than about 9 hours prior to the radiation and/surgery. In some embodiments, the nanoparticle composition is administered less than about any of 1, 2, 3, 4, 5, 6, 7, 8, 9, or 10 days prior to the radiation/surgery. In some embodiments, the VEGFR-2 specific inhibitor is administered less than about any of 1, 3, 6, 9, 12, 18, 24, 48, 60, 72, 84, 96, 108, or 120 hours after the radiation and/or surgery. In some embodiments, it may be desirable to extend the time period for treatment significantly, where several days to several weeks lapse between the two therapies.

Radiation contemplated herein includes, for example, y-rays, X-rays (external beam), and the directed delivery of radioisotopes to tumor cells. Other forms of DNA damaging factors are also contemplated such as microwaves and UV irradiation are also contemplated. Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may comprise fractionated individual doses of 2 Gy. Dosage ranges for radioisotopes vary widely, and depends on the half-life of the isotope and the strength and type of radiation emitted.

When the radiation comprises use of radioactive isotopes, the isotope may be conjugated to a targeting agent, such as a therapeutic antibody, which carries the radionucleotide to the target tissue. Suitable radioactive isotopes include, but are not limited to, astatine²¹¹, ¹⁴carbon, ⁵¹chromium, ³⁶chlorine, ⁵⁸cobalt, copper⁶⁷, ¹⁵²Eu, gallium⁶⁷, ³hydrogen, iodine¹²³, iodine¹³¹, indium¹¹¹, ⁵⁷iron, ⁵⁹iron, ³²phosphorus, rhenium¹⁸⁶, ⁷⁵selenium, ³⁵sulphur, technicium⁹⁹, and/or yttrium⁹⁰.

In some embodiments, enough radiation is applied to the individual so as to allow reduction of the normal dose of the VEGFR-2 specific inhibitor required to effect the same degree of treatment by at least about any of 5%, 10%, 20%, 30%, 50%, 60%, 70%, 80%, 90%, or more. In some embodiments, enough VEGFR-2 specific inhibitor is administered so as to allow reduction of the normal dose of the radiation required to effect the same degree of treatment by at least about any of 5%, 10%, 20%, 30%, 50%, 60%, 70%, 80%, 90%, or more. In some embodiments, the dose of both the VEGFR-2 specific inhibitor and the radiation are reduced as compared to the corresponding normal dose of each when used alone.

In some embodiments, the combination of administration of the VEGFR-2 specific inhibitor and the radiation therapy produce supra-additive effect. In some embodiments, the VEGFR-2 specific inhibitor is administered once, and the radiation is applied five times at 80 Gy daily.

Surgery described herein includes resection in which all or part of cancerous tissue is physically removed, exercised, and/or destroyed. Tumor resection refers to physical removal of at least part of a tumor. In addition to tumor resection, treatment by surgery includes laser surgery, cryosurgery, electrosurgery, and micropically controlled surgery (Mohs surgery). Removal of superficial surgery, precancers, or normal tissues are also contemplated.

The radiation therapy and/or surgery may be carried out in addition to the administration of chemotherapeutic agents. For example, the individual may first be administered with a taxane-containing nanoparticle composition and at least one other chemotherapeutic agent, and subsequently be subject to radiation therapy and/or surgery. Alternatively, the individual may first be treated with radiation therapy and/or surgery, which is then followed by the administration of a nanoparticle composition and at least one other chemotherapeutic agent. Other combinations are also contemplated.

In one embodiment, the present invention can be used for increasing the duration of survival of a human patient susceptible to or diagnosed with metastatic cancer. Duration of survival is defined as the time from first administration of the drug to death. In a preferred aspect, a VEGFR-2 specific inhibitor is administered to the human patient in combination with one or more chemotherapeutic agents, thereby the duration of survival of the patient is effectively increased as compared to a chemotherapy alone. For example, patient group treated with the VEGFR-2 specific inhibitor combined with a chemotherapeutic cocktail of at least two, preferably three, chemotherapeutic agents may have a median duration of survival that is at least about 2 months, preferably between about 2 and about 5 months, longer than that of the patient group treated with the same chemotherapeutic cocktail alone, said increase being statistically significant. Duration of survival can also be measured by stratified hazard ratio (HR) of the treatment group versus control group, which represents the risk of death for a patient during the treatment. Preferably, a combination treatment of VEGFR-2 specific inhibitor and one or more chemotherapeutic agents significantly reduces the risk of death by at least about 30% (i.e., a stratified HR of about 0.70), preferably by at least about 35% (i.e., a stratified HR of about 0.65), when compared to a chemotherapy alone.

In another embodiment, the present invention provides methods for increasing progression free survival of a human patient susceptible to or diagnosed with metastatic cancer. Time to disease progression, is defined as the time from administration of the drug until disease progression. In a preferred embodiment, the combination treatment of the invention using VEGFR-2 specific inhibitor and one or more chemotherapeutic agents significantly increases progression free survival by at least about 2 months, preferably by about 2 to about 5 months, when compared to a treatment with chemotherapy alone.

In yet another embodiment, the treatment of the present invention significantly increases response rate in a group of human patients susceptible to or diagnosed with metastatic cancer who are treated with various therapeutics. Response rate is defined as the percentage of treated patients who responded to the treatment. In one aspect, the combination treatment of the invention using VEGFR-2 specific inhibitor and one or more chemotherapeutic agents significantly increases response rate in the treated patient group compared to the group treated with chemotherapy alone, said increase having a Chi-square p-value of less than 0.005.

In one aspect, the present invention provides methods for increasing duration of response in a human patient or a group of human patients susceptible to or diagnosed with a cancer. Duration of response is defined as the time from the initial response to disease progression. In a combination treatment of the invention using VEGFR-2 specific inhibitor and one or more chemotherapeutic agents, a statistically significant increase of at least 2 months in duration of response is obtainable and preferred.

VEGFR-2 Specific Inhibitors

VEGFR-2 specific inhibitors were generated as described in U.S. patent application Ser. Nos. 11/482,641, 11/448,171, and PCT International Application Publication No. WO 05/056764, which are hereby incorporated by reference. These inhibitors are further described in U.S. Provisional Pat. Application No. 60/899,094 which is hereby incorporated by reference.

Sequences of the preferred VEGFR-2 binding ¹⁰Fn3 polypeptides useful for the invention are as follows:

SEQ ID NO: 2

VSDVPRDLEVVAATPTSLLISWRHPHFPTRYYRITYGETGGNSPVQEFTV
PLQPPTATISGLKPGVDYTITVYAVTEGPNERSLFIPISINYRT

SEQ ID NO: 3

EVVAATPTSLLISWRHPHFPTRYYRITYGETGGNSPVQEFTVPLQPPTA
TISGLKPGVDYTITVYAVTEGPNERSLFIPISINYRT

SEQ ID NO: 4

GEVVAATPTSLLISWRHPHFPTRYYRITYGETGGNSPVQEFTVPLQPP
TATISGLKPGVDYTITVYAVTDGRNGRLLSIPISINYRTEIDKPCQ

SEQ ID NO: 5

EVVAATPTSLLISWRHPHFPTRYYRITYGETGGNSPVQEFTVPLQPPTA
TISGLKPGVDYTITVYAVTDGRNGRLLSIPISINYRT

SEQ ID NO: 6

EVVAATPTSLLISWRHPHFPTRYYRITYGETGGNSPVQEFTVPLQPPTA
TISGLKPGVDYTITGYAVTMGLYGHELLTPISINYRT

SEQ ID NO: 7

EVVAATPTSLLISWRHPHFPTRYYRITYGETGGNSPVQEFTVPLQPPTA
TISGLKPGVDYTITGYAVTDGENGQFLLVPISINYRT

SEQ ID NO: 8

EVVAATPTSLLISWRHPHFPTRYYRITYGETGGNSPVQEFTVPLQPPTA
TISGLKPGVDYTITGYAVTMGPNDNELLTPISINYRT

SEQ ID NO: 9

EVVAATPTSLLISWRHPHFPTRYYRITYGETGGNSPVQEFTVPLQPPTA
TISGLKPGVDYTITGYAVTAGWDDHELFIPISINYRT

SEQ ID NO: 10

EVVAATPTSLLISWRHPHFPTRYYRITYGETGGNSPVQEFTVPLQPPTA
TISGLKPGVDYTITGYAVTSGHNDHMLMIPISINYRT

SEQ ID NO: 11

EVVAATPTSLLISWRHPHFPTRYYRITYGETGGNSPVQEFTVPLQPPTA
TISGLKPGVDYTITGYAVTAGYNDQILMTPISINYRT

SEQ ID NO: 12

EVVAATPTSLLISWRHPHFPTRYYRITYGETGGNSPVQEFTVPLQPPTA
TISGLKPGVDYTITGYAVTFGLYGKELLIPISINYRT

SEQ ID NO: 13

EVVAATPTSLLISWRHPHFPTRYYRITYGETGGNSPVQEFTVPLQPPTA
TISGLKPGVDYTITGYAVTTGPNDRLLFVPISINYRT

SEQ ID NO: 14

EVVAATPTSLLISWRHPHFPTRYYRITYGETGGNSPVQEFTVPLQPPTA
TISGLKPGVDYTITGYAVTDVYNDHEIKTPISINYRT

SEQ ID NO: 15

EVVAATPTSLLISWRHPHFPTRYYRITYGETGGNSPVQEFTVPLQPPTA
TISGLKPGVDYTITGYAVTDGKDGRVLLTPISINYRT

SEQ ID NO: 16

EVVAATPTSLLISWRHPHFPTRYYRITYGETGGNSPVQEFTVPLQPPTA
TISGLKPGVDYTITGYAVTEVHHDREIKTPISINYRT

SEQ ID NO: 17

EVVAATPTSLLISWRHPHFPTRYYRITYGETGGNSPVQEFTVPLQPPTA
TISGLKPGVDYTITGYAVTQAPNDRVLYTPISINYRT

SEQ ID NO: 18

EVVAATPTSLLISWRHPHFPTRYYRITYGETGGNSPVQEFTVPLQPPTA
TISGLKPGVDYTITGYAVTREENDHELLIPISINYRT

SEQ ID NO: 19

EVVAATPTSLLISWRHPHFPTRYYRITYGETGGNSPVQEFTVPLQPPTA
TISGLKPGVDYTITGYAVTVTHNGHPLMTPISINYRT

SEQ ID NO: 20

EVVAATPTSLLISWRHPHFPTRYYRITYGETGGNSPVQEFTVPLQPPTA
TISGLKPGVDYTITGYAVTLALKGHELLTPISINYRT

SEQ ID NO: 21

VSDVPRDLEVVAATPTSLLISWRHPHFPTRYYRITYGETGGNSPVQEF
TVPLQPPTATISGLKPGVDYTITGYAVTVAQNDHELITPISINYRT

SEQ ID NO: 22

VSDVPRDL/QEVVAATPTSLLISWRHPHFPTRYYRITYGETGGNSPVQE
FTVPLQPPAATISGLKPGVDYTITGYAVTMAQSGHELFTPISINYRT

SEQ ID NO: 24

EVVAATPTSLLISWRHPHFPTRYYRITYGETGGNSPVQEFTVPLQPPTA
TISGLKPGVDYTITGYAVTVERNGRVLMTPISINYRT

SEQ ID NO: 25

EVVAATPTSLLISWRHPHFPTRYYRITYGETGGNSPVQEFTVPLQPPTA
TISGLKPGVDYTITGYAVTVERNGRHLMTPISINYRT

SEQ ID NO: 26

EVVAATPTSLLISWRHPHFPTRYYRITYGETGGNSPVQEFTVPLQPPTA
TISGLKPGVDYTITGYAVTLERNGRELMTPISINYRT

SEQ ID NO: 27

EVVAATPTSLLISWRHPHFPTRYYRITYGETGGNSPVQEFTVPLQPPTA
TISGLKPGVDYTITGYAVTEERNGRTLRTPISINYRT

SEQ ID NO: 28

EVVAATPTSLLISWRHPHFPTRYYRITYGETGGNSPVQEFTVPLQPPTA
TISGLKPGVDYTITGYAVTVERNDRVLFTPISINYRT

SEQ ID NO: 29

EVVAATPTSLLISWRHPHFPTRYYRITYGETGGNSPVQEFTVPLQPPTA
TISGLKPGVDYTITGYAVTVERNGRELMTPISINYRT

SEQ ID NO: 30

EVVAATPTSLLISWRHPHFPTRYYRITYGETGGNSPVQEFTVPLQPPTA
TISGLKPGVDYTITGYAVTLERNGRELMVPISINYRT

SEQ ID NO: 31

EVVAATPTSLLISWRHPHFPTRYYRITYGETGGNSPVQEFTVPLQPPTA
TISGLKPGVDYTITGYAVTDGRNDRKLMVPISINYRT

SEQ ID NO: 32

EVVAATPTSLLISWRHPHFPTRYYRITYGETGGNSPVQEFTVPLQPPTA
TISGLKPGVDYTITGYAVTDGQNGRLLNVPISINYRT

SEQ ID NO: 33

EVVAATPTSLLISWRHHPHFPTRYYRITYGETGGNSPVQEFTVPLQPPT
ATISGLKPGVDYTITGYAVTVHWNGRELMTPISINYRT

SEQ ID NO: 34

EVVAATPTSLLISWRHPHFPTRYYRITYGETGGNSPVQEFTVPLQPPTA
TISGLKPGVDYTITGYAVTEEWNGRVLMTPISINYRT

SEQ ID NO: 35

EVVAATPTSLLISWRHPHFPTRYYRITYGETGGNSPVQEFTVPLQPPTA
TISGLKPGVDYTITGYAVTVERNGHTLMTPISINYRT

SEQ ID NO: 36

EVVAATPTSLLISWRHPHFPTRYYRITYGETGGNSPVQEFTVPLQPPTA
TISGLKPGVDYTITGYAVTVEENGRQLMTPISINYRT

SEQ ID NO: 37

EVVAATPTSLLISWRHPHFPTRYYRITYGETGGNSPVQEFTVPLQPPTA
TISGLKPGVDYTITGYAVTLERNGQVLFTPISINYRT

SEQ ID NO: 38

EVVAATPTSLLISWRHPHFPTRYYRITYGETGGNSPVQEFTVPLQPPTA
TISGLKPGVDYTITGYAVTVERNGQVLYTPISINYRT

SEQ ID NO: 39

EVVAATPTSLLISWRHPHFPTRYYRITYGETGGNSPVQEFTVPLQPPTA
TISGLKPGVDYTITGYAVTWGYKDHELLIPISINYRT

SEQ ID NO: 40

EVVAATPTSLLISWRHPHFPTRYYRITYGETGGNSPVQEFTVPLQPPTA
TISGLKPGVDYTITGYAVTLGRNDRELLTPISINYRT

SEQ ID NO: 41

EVVAATPTSLLISWRHPHFPTRYYRITYGETGGNSPVQEFTVPLQPPTA
TISGLKPGVDYTITGYAVTDGPNDRLLNIPISINYRT

SEQ ID NO: 42

EVVAATPTSLLISWRHPHFPTRYYRITYGETGGNSPVQEFTVPLQPPTA
TISGLKPGVDYTITGYAVTFARDGHEILTPISINYRT

SEQ ID NO: 43

EVVAATPTSLLISWRHPHFPTRYYRITYGETGGNSPVQEFTVPLQPPTA
TISGLKPGVDYTITGYAVTLEQNGRELMTPISINYRT

SEQ ID NO: 44

EVVAATPTSLLISWRHPHFPTRYYRITYGETGGNSPVQEFTVPLQPPTA
TISGLKPGVDYTITGYAVTVEENGRVLNTPISINYRT

SEQ ID NO: 45

EVVAATPTSLLISWRHPHFPTRYYRITYGETGGNSPVQEFTVPLQPPTA
TISGLKPGVDYTITGYAVTLEPNGRYLMVPISINYRT

SEQ ID NO: 46

EVVAATPTSLLISWRHPHFPTRYYRITYGETGGNSPVQEFTVPLQPPTA
TISGLKPGVDYTITGYAVTEGRNGRELFIPISINYRT

SEQ ID NO: 47

VSDVPRDLEVVAATPTSLLISWRHPHFPTRYYRITYGETGGNSPVQEF
TVPLQPPAATISGLKPGVDYTITGYAVTWERNGRELFTPISINYRT

SEQ ID NO: 48

VSDVPRDLEVVAATPTSLLISWRHPHFPTRYYRITYGETGGNSPVQEF
TVPLQPPAATISGLKPGVDYTITGYAVTKERNGRELFTPISINYRT

SEQ ID NO: 49

VSDVPRDLEVVAATPTSLLISWRHPHFPTHYYRITYGETGGNSPVQEF
TVPLQPPAATISGLKPGVDYTITGYAVTTERTGRELFTPISINYRT

SEQ ID NO: 50

VSDVPRDLEVVAATPTSLLISWRHPHFPTHYYRITYGETGGNSPVQEF
TVPLQPPAATISGLKPGVDYTITGYAVTKERSGRELFTPISINYRT

SEQ ID NO: 51

VSDVPRDLEVVAATPTSLLISWRHPHFPTHYYRITYGETGGNSPVQEF
TVPLQPPAATISGLKPGVDYTITGYAVTLERDGRELFTPISINYRT

SEQ ID NO: 52

VSDVPRDLEVVAATPTSLLISWRHPHFPTRYYRITYGETGGNSPVQEF
TVPLQPPLATISGLKPGVDYTITG/VYAVTKERNGRELFTPISINYRT

SEQ ID NO: 53

VSDVPRDLEVVAATPTSLLISWRHPHFPTRYYRITYGETGGNSPVQEF
TVPLQPTTATISGLKPGVDYTITGYAVTWERNGRELFTPISINYRT

SEQ ID NO: 54

VSDVPRDLEVVAATPTSLLISWRHPHFPTRYYRITYGETGGNSPVQEF
TVPLQPTVATISGLKPGVDYTITGYAVTLERNDRELFTPISINYRT

SEQ ID NO: 55

MGEVVAATPTSLLISWRHPHFPTRYYRITYGETGGNSPVQEFTVPLQP
PTATISGLKPGVDYTITVYAVTDGRNGRLLSIPISINYRTEIDKPSQ

SEQ ID NO: 56

MGEVVAATPTSLLISWRHPHFPTRYYRITYGETGGNSPVQEFTVPLQP
PTATISGLKPGVDYTITVYAVTDGRNGRLLSIPISINYRTEIDKPCQ

SEQ ID NO: 57

MVSDVPRDLEVVAATPTSLLISWRHPHFPTRYYRITYGETGGNSPVQE
FTVPLQPPTATISGLKPGVDYTITVYAVTDGRNGRLLSIPISINYRTEID
KPSQ

SEQ ID NO: 58

MGEVVAATPTSLLISWRHPHFPTRYYRITYGETGGNSPVQEFTVPLQP
PTATISGLKPGVDYTITVYAVTDGWNGRLLSIPISINYRT

SEQ ID NO: 59

MGEVVAATPTSLLISWRHPHFPTRYYRITYGETGGNSPVQEFTVPLQP
PTATISGLKPGVDYTITVYAVTEGPNERSLFIPISINYRT

SEQ ID NO: 60

MVSDVPRDLEVVAATPTSLLISWRHPHFPTRYYRITYGETGGNSPVQE
FTVPLQPPTATISGLKPGVDYTITVYAVTEGPNERSLFIPISINYRT

Proteins of the invention include the disclosed amino acid sequences with deletions of the first 8 amino acids and may include additional amino acids at the N- or C-termini. For example, an additional MG sequence may be placed at the N-terminus. The M will usually be cleaved off, leaving a GEV . . . sequence at the N-terminus. The re-addition of the normal 8 amino acids at the N-terminus also produces a VEGFR-2 binding protein with desirable properties. In some embodiments, the N-terminal methionine is cleaved off. For use in vivo, a form suitable for pegylation may be generated. In one embodiment, a C-terminal tail comprising a cysteine can be added (for example, EIDKPCQ (SEQ ID NO:61) is added at the C-terminus).

Additional Protein Embodiments

Proteins useful in methods of the invention include a single domain polypeptide as described herein, generally a polypeptide that binds to a target, such as VEGFR-2, and where target binding activity situated within a single structural domain, as differentiated from, for example, antibodies and single chain antibodies, where antigen binding activity is generally contributed by both a heavy chain variable domain and a light chain variable domain. The disclosure also provides larger proteins that may comprise single domain polypeptides that bind to target. For example, a plurality of single domain polypeptides may be connected to create a composite molecule with increased avidity or multivalency. Likewise, a single domain polypeptide may be attached (e.g., as a fusion protein) to any number of other polypeptides. In certain aspects a single domain polypeptide may comprise at least five to seven beta or beta-like strands distributed among at least two beta sheets, as exemplified by immunoglobulin and immunoglobulin-like domains. A beta-like strand is a string of amino acids that participates in the stabilization of a single domain polypeptide but does not necessarily adopt a beta strand conformation. Whether a beta-like strand participates in the stabilization of the protein may be assessed by deleting the string or altering the sequence of the string and analyzing whether protein stability is diminished. Stability may be assessed by, for example, thermal denaturation and renaturation studies. Preferably, a single domain polypeptide will include no more than two beta-like strands. A beta-like strand will not usually adopt an alpha-helical conformation but may adopt a random coil structure. In the context of an immunoglobulin domain or an immunoglobulin-like domain, a beta-like strand will most often occur at the position in the structure that would otherwise be occupied by the most N-terminal beta strand or the most C-terminal beta strand. An amino acid string which, if situated in the interior of a protein sequence would normally form a beta strand, may, when situated at a position closer to an N- or C-terminus, adopt a conformation that is not clearly a beta strand and is referred to herein as a beta-like strand.

In certain embodiments, the disclosure provides single domain polypeptides that bind to VEGFR-2. Preferably the single domain polypeptides bind to human VEGFR-2 or a model species VEGFR-2. A single domain polypeptide may comprise between about 80 and about 150 amino acids that have a structural organization comprising: at least seven beta strands or beta-like strands distributed between at least two beta sheets, and at least one loop portion connecting two beta strands or beta-like strands, which loop portion participates in binding to VEGFR-2. In other words a loop portion may link two beta strands, two beta-like strands or one beta strand and one beta-like strand. Typically, one or more of the loop portions will participate in VEGFR-2 binding, although it is possible that one or more of the beta or beta-like strand portions will also participate in VEGFR-2 binding, particularly those beta or beta-like strand portions that are situated closest to the loop portions.

A single domain polypeptide may comprise a structural unit that is an immunoglobulin domain or an immunoglobulin-like domain. A single domain polypeptide may bind to any part of VEGFR-2, although polypeptides that bind to an extracellular domain of a VEGFR-2 are preferred. Binding may be assessed in terms of equilibrium constants (e.g., dissociation, K_{d}) and in terms of kinetic constants (e.g., on rate constant, kₒₙ and off rate constant, k_{off}). A single domain polypeptide will typically be selected to bind to VEGFR-2 with a K_{d} of less than about 10⁻⁶ M, or less than about 10⁻⁷ M, about 5×10⁻⁸ M, about 10⁻⁸ M or less than about 10⁻⁹ M. VEGFR-2 binding polypeptides may compete for binding with one, or two or more members of the VEGF family, particularly VEGF-A, VEGF-C, and/or VEGF-D, and may inhibit one or more VEGFR-2-mediated biological events, such as proliferation of cancer cells and cancer metastasis. VEGFR-2 binding polypeptides may be used for therapeutic purposes as well as for any purpose involving the detection or binding of VEGFR-2. Polypeptides for therapeutic use will generally have a K_{d} of less than 5×10⁻⁸ M, less than 10⁻⁸ M or less than 10⁻⁹ M, although higher K_{d} values may be tolerated where the k_{off} is sufficiently low or the kₒₙ is sufficiently high.

In certain embodiments, the single domain polypeptide comprises an immunoglobulin (Ig) variable domain. The Ig variable domain may, for example, be selected from the group consisting of: a human V_{L} domain, a human V_{H} domain and a camelid V_{HH} domain. One, two, three or more loops of the Ig variable domain may participate in binding to VEGFR-2, and typically any of the loops known as CDR1, CDR2 or CDR3 will participate in VEGFR-2 binding.

In certain embodiments, the single domain polypeptide comprises an immunoglobulin-like domain. One, two, three or more loops of the immunoglobulin-like domain may participate in binding to VEGFR-2. A preferred immunoglobulin-like domain is a fibronectin type III (Fn3) domain. Such domain may comprise, in order from N-terminus to C-terminus, a beta or beta-like strand, A; a loop, AB; a beta strand, B; a loop, BC; a beta strand C; a loop CD; a beta strand D; a loop DE; a beta strand F; a loop FG; and a beta or beta-like strand G.

Optionally, any or all of loops AB, BC, CD, DE, EF and FG may participate in VEGFR-2 binding, although preferred loops are BC, DE and FG. A preferred Fn3 domain is an Fn3 domain derived from human fibronectin, particularly the 10^{th} Fn3 domain of fibronectin, referred to as ¹⁰Fn3. It should be noted that none of VEGFR-2 binding polypeptides disclosed herein have an amino acid sequence that is identical to native ¹⁰Fn3; the sequence has been modified to obtain VEGFR-2 binding proteins, but proteins having the basic structural features of ¹⁰Fn3, and particularly those retaining recognizable sequence homology to the native ¹⁰Fn3 are nonetheless referred to herein as “¹⁰Fn3 polypeptides”. This nomenclature is similar to that found in the antibody field where, for example, a recombinant antibody V_{L} domain generated against a particular target protein may not be identical to any naturally occurring V_{L} domain but nonetheless the protein is recognizably a V_{L} protein. A ¹⁰Fn3 polypeptide may be at least 60%, 65%, 70%, 75%, 80%, 85%, or 90% identical to the human ¹⁰Fn3 domain, shown in SEQ ID NO:1. Much of the variability will generally occur in one or more of the loops. Each of the beta or beta-like strands of a ¹⁰Fn3 polypeptide may consist essentially of an amino acid sequence that is at least 80%, 85%, 90%, 95% or 100% identical to the sequence of a corresponding beta or beta-like strand of SEQ ID NO:1, provided that such variation does not disrupt the stability of the polypeptide in physiological conditions. A ¹⁰Fn3 polypeptide may have a sequence in each of the loops AB, CD, and EF that consists essentially of an amino acid sequence that is at least 80%, 85%, 90%, 95% or 100% identical to the sequence of a corresponding loop of SEQ ID NO:1. In many instances, any or all of loops BC, DE, and FG will be poorly conserved relative to SEQ ID NO:1. For example, all of loops BC, DE, and FG may be less than 20%, 10%, or 0% identical to their corresponding loops in SEQ ID NO:1.

In certain embodiments, the disclosure provides a non-antibody polypeptide comprising a domain having an immunoglobulin-like fold that binds to VEGFR-2. The non-antibody polypeptide may have a molecular weight of less than 20 kD, or less than 15 kD and will generally be derived (by, for example, alteration of the amino acid sequence) from a reference, or “scaffold”, protein, such as an Fn3 scaffold. The non-antibody polypeptide may bind VEGFR-2 with a IQ less than 10⁻⁶ M, or less than 10⁻⁷ M, less than 5×10⁻⁸ M, less than 10⁻⁸ M or less than 10⁻⁹ M. The unaltered reference protein either will not meaningfully bind to VEGFR-2 or will bind with a K_{d} of greater than 10⁻⁶ M. The non-antibody polypeptide may inhibit VEGFR-2 signaling, particularly where the non-antibody polypeptide has a K_{d} of less than 5×10⁻⁸ M, less than 10⁻⁸ M or less than 10⁻⁹ M, although higher K_{d} values may be tolerated where the k_{off} is sufficiently low (e.g., less than 5×10⁻⁴ s⁻¹). The immunoglobulin-like fold may be a ¹⁰Fn3 polypeptide.

In certain embodiments, the disclosure provides a polypeptide comprising a single domain having an immunoglobulin fold that binds to VEGFR-2. The polypeptide may have a molecular weight of less than 20 kD, or less than 15 kD and will generally be derived (by, for example, alteration of the amino acid sequence) from a variable domain of an immunoglobulin. The polypeptide may bind VEGFR-2 with a K_{d} less than 10⁻⁶ M, or less than 10⁻⁷ M, less than 5×10⁻⁸M, less than 10⁻⁸ M or less than 10⁻⁹ M. The polypeptide may inhibit VEGFR-2 signaling, particularly where the polypeptide has a K_{d} of less than 5×10⁻⁸ M, less than 10⁻⁸ M or less than 10⁻⁹ M, although higher K_{d} values may be tolerated where the k_{off} is sufficiently low or where the kₒₙ is sufficiently high. In certain preferred embodiments, a single domain polypeptide having an immunoglobulin fold derived from an immunoglobulin light chain variable domain and capable of binding to VEGFR-2 may comprise an amino acid sequence selected from the group consisting of: those disclosed in the tables. In some embodiments, the polypeptide comprises an amino acid sequence that is at least 80% identical to SEQ NO:1. In some embodiments, the polypeptide comprises an amino acid sequence selected from the group consisting of any of SEQ ID NOs:2-60. In some embodiments, the polypeptide further comprises PEG.

In certain preferred embodiments, the disclosure provides VEGFR-2 binding polypeptides comprising the amino acid sequence of any of disclosed in the tables with the most desirable biochemical features. In the case of a polypeptide comprising such amino acid sequences, a PEG moiety or other moiety of interest, may be covalently bound to the cysteine at position from about 85 to 100 depending on the protein. The PEG moiety may also be covalently bonded to an amine moiety in the polypeptide. The amine moiety may be, for example, a primary amine found at the N-terminus of a polypeptide or an amine group present in an amino acid, such as lysine or arginine. In certain embodiments, the PEG moiety is attached at a position on the polypeptide selected from the group consisting of: a) the N-terminus; b) between the N-terminus and the most N-terminal beta strand or beta-like strand; c) a loop positioned on a face of the polypeptide opposite the target-binding site; d) between the C-terminus and the most C-terminal beta strand or beta-like strand; and e) at the C-terminus.

In certain aspects, the disclosure provides short peptide sequences that mediate VEGFR-2 binding. Such sequences may mediate VEGFR-2 binding in an isolated form or when inserted into a particular protein structure, such as an immunoglobulin or immunoglobulin-like domain. Examples of such sequences include those disclosed (such as SEQ ID NOs:2-60) and other sequences that are at least 85%, 90%, or 95% identical to SEQ ID NO:1 to such sequences and retain VEGFR-2 binding activity. Accordingly, the disclosure provides substantially pure polypeptides comprising an amino acid sequence that is at least 85% identical to the sequence of any of such sequences, wherein said polypeptide binds to a VEGFR-2 and competes with an VEGF species for binding to VEGFR-2. Examples of such polypeptides include a polypeptide comprising an amino acid sequence that is at least 80%, 85%, 90%, 95% or 100% identical to an amino acid sequence of SEQ ID:2-60. Preferably such polypeptides will inhibit a biological activity of a VEGF and may bind to VEGFR-2 with a K_{d} less than 10⁻⁶ M, or less than 10⁻⁷ M, less than 5×10⁻⁸ M, less than 10⁻⁸ M or less than 10⁻⁹ M.

In certain embodiments, any of the VEGFR-2 binding polypeptides described herein may be bound to one or more additional moieties, including, for example, a moiety that also binds to VEGFR-2 (e.g., a second identical or different VEGFR-2 binding polypeptide), a moiety that binds to a different target (e.g., to create a dual-specificity binding agent), a labeling moiety, a moiety that facilitates protein purification or a moiety that provides improved pharmacokinetics. Improved pharmacokinetics may be assessed according to the perceived therapeutic need. Often it is desirable to increase bioavailability and/or increase the time between doses, possibly by increasing the time that a protein remains available in the serum after dosing. In some instances, it is desirable to improve the continuity of the serum concentration of the protein over time (e.g., decrease the difference in serum concentration of the protein shortly after administration and shortly before the next administration). Moieties that tend to slow clearance of a protein from the blood include polyethylene glycol, sugars (e.g., sialic acid), and well-tolerated protein moieties (e.g., Fc fragment or serum albumin). The single domain polypeptide may be attached to a moiety that reduces the clearance rate of the polypeptide in a mammal (e.g., mouse, rat, or human) by greater than three-fold relative to the unmodified polypeptide. Other measures of improved pharmacokinetics may include serum half-life, which is often divided into an alpha phase and a beta phase. Either or both phases may be improved significantly by addition of an appropriate moiety. Where polyethylene glycol is employed, one or more PEG molecules may be attached at different positions in the protein, and such attachment may be achieved by reaction with amines, thiols or other suitable reactive groups. Pegylation may be achieved by site-directed pegylation, wherein a suitable reactive group is introduced into the protein to create a site where pegylation preferentially occurs. In a preferred embodiment, the protein is modified so as to have a cysteine residue at a desired position, permitting site directed pegylation on the cysteine. PEG may vary widely in molecular weight and may be branched or linear. Notably, the present disclosure establishes that pegylation is compatible with target binding activity of ¹⁰Fn3 polypeptides and, further, that pegylation does improve the pharmacokinetics of such polypeptides. Accordingly, in one embodiment, the disclosure provides pegylated forms of ¹⁰Fn3 polypeptides, regardless of the target that can be bound by such polypeptides.

Additional Bispecific and Multi-Specific Embodiments

In many embodiments it will be desirable to make multi-specific compositions, e.g., compositions that bind more than one target or other protein of interest.

In one aspect, proteins useful in the methods of the invention comprise a first protein with a binding affinity of about 10 nM (other appropriate affinity described herein) to first desired target (e.g., VEGFR-2) or less and binds an undesired, related target (e.g., VEGFR-1 and VEGFR-3) with a binding affinity of about 1 μM (other appropriate affinity described herein) or greater and is preferably a single domain or substantially monovalent and is linked to attached to a second protein with a binding affinity of about 10 nM (other appropriate affinity described herein) to a second desired target (e.g., c-kit, Her-2, FGFR-1, VEGFR-1, VEGF-A, VEGF-C, VEGF-D, folate receptor, c-Met, EGFR) or less and binds an undesired, related target (e.g., human insulin receptor) with a binding affinity of about 1 μM (other appropriate affinity described herein) or greater and is preferably a single domain or substantially monovalent. Such molecules with bispecific affinity can be further attached to other molecules, including other proteins described herein.

Additional PEG Embodiments

In some embodiments of the methods of treatments, VEGFR-2 specific inhibitors are fibronectin based scaffolds, such as Adnectins™, with engineering of Cys or Lys amino acids.

PEG (or functionally similar molecule) is used to connect two proteins that are non-antibody moieties that bind a two or more different targets or protein of interest (e.g., a PK modulating protein), particularly proteins wherein each binding protein is comprised of a single domain or multiple domains, usually wherein each domain is about 50 or about 60 or about 75 amino acids or more (as opposed to small peptides of 5 to 20 amino acids). Preferably fibronectin based scaffolds, such as Adnectins™, can be used advantageously in such embodiments and more preferably with the proper engineering of Cys or Lys amino acids.

In addition, nonPEG and PEG aspects of the VEGFR-2 specific inhibitors include antibody moieties (e.g., camel antibodies and their derivatives, as well as single chain and domain antibodies; and particularly those expressed from microbes) and antibody-like moieties (e.g., derivatives of lipocalins, ankyrins, multiple Cys-Cys domains, and tetranectins; and particularly those expressed from microbes), particularly those less than about 40 kD that are connect by PEG, and more particularly those that have a limited number of Cys amino acids.

There are many properties and advantages of PEG linked proteins. When such proteins are expressed in microbes it may be preferable to isolate domains and them link them via PEG or other polymeric linker. PEG, or other functionally operably polymeric linker, can be used to optimally vary the distance between each protein moiety to create a protein with one or more of the following characteristics: 1) reduced or increased steric hindrance of binding of one or more protein domain when binding to a protein of interest (e.g., a target), 2) connect two or more domains that bind different targets, 3) increase protein stability or solubility without searching for additional amino acid substitutions to increase stability or solubility (e.g., solubility at least about 20 mg/mL, or at least about 50 mg/mL), 4) decrease protein aggregation without searching for additional amino acid substitutions to decrease stability (e.g., as measured by SEC), 4) increase the overall avidity or affinity of the protein for the protein of interest by adding additional binding domains. Additional advantages of PEG linked proteins include rapidly making monospecific, multi-valent binding modes, as well as multi-specific, monovalent or multivalent binding modes depending on the number of protein targeting moieties that are included in the PEG linked protein.

¹⁰Fn3 polypeptides useful for methods of the invention can be pegylated and retain ligand binding activity. In a preferred embodiment, the pegylated ¹⁰Fn3 polypeptide is produced by site-directed pegylation, particularly by conjugation of PEG to a cysteine moiety at the N- or C-terminus. Accordingly, the present disclosure provides a target-binding ¹⁰Fn3 polypeptide with improved pharmacokinetic properties, the polypeptide comprising: a ¹⁰Fn3 domain having from about 80 to about 150 amino acids, wherein at least one of the loops of said ¹⁰Fn3 domain participate in target binding; and a covalently bound PEG moiety, wherein said ¹⁰Fn3 polypeptide binds to the target with a K_{d} of less than 100 nM and has a clearance rate of less than 30 mL/hr/kg in a mammal. The PEG moiety may be attached to the ¹⁰Fn3 polypeptide by site directed pegylation, such as by attachment to a Cys residue, where the Cys residue may be positioned at the N-terminus of the ¹⁰Fn3 polypeptide or between the N-terminus and the most N-terminal beta or beta-like strand or at the C-terminus of the ¹⁰Fn3 polypeptide or between the C-terminus and the most C-terminal beta or beta-like strand. A Cys residue may be situated at other positions as well, particularly any of the loops that do not participate in target binding. A PEG moiety may also be attached by other chemistry, including by conjugation to amines. In addition, the invention includes this type of N or C terminal PEG conjugation to antibody moieties (e.g., camel antibodies and their derivatives, as well as single chain and domain antibodies; and particularly those expressed from microbes) and antibody-like moieties (e.g., derivatives of lipocalins, ankyrins, multiple Cys-Cys domains, and tetranectins; and particularly those expressed from microbes), particularly those less than 40 kD that are connect by PEG, and more particularly those that have a limited number of Cys amino acids.

In one specific embodiment of the present invention, modified forms of the subject soluble polypeptides comprise linking the subject soluble polypeptides to nonproteinaceous polymers. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. Examples of the modified polypeptide of the invention include PEGylated proteins further described herein.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula:

X—O(CH₂CH₂O)ₙ₋₁CH₂CH₂OH (1)

where n is 20 to 2300 and X is H or a terminal modification, e.g., a C₁₋₄ alkyl. In one embodiment, the PEG of the invention terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, European Pat. No. EP-A 0473084 and U.S. Pat. No. 5,932,462. One form of PEGs includes two PEG side-chains (PEG2) linked via the primary amino groups of a lysine (Monfardini, C. et al. Bioconjugate Chem. 6 (1995) 62-69).

In a preferred embodiment, a pegylated ¹⁰Fn3 polypeptide is produced by site-directed pegylation, particularly by conjugation of PEG to a cysteine moiety at the N- or C-terminus. Accordingly, the present disclosure provides a target-binding ¹⁰Fn3 polypeptide with improved pharmacokinetic properties, the polypeptide comprising: a ¹⁰Fn3 domain having from about 80 to about 150 amino acids, wherein at least one of the loops of said ¹⁰Fn3 domain participate in target binding; and a covalently bound PEG moiety, wherein said ¹⁰Fn3 polypeptide binds to the target with a K_{d} of less than 100 nM and has a clearance rate of less than 30 mL/hr/kg in a mammal. The PEG moiety may be attached to the ¹⁰Fn3 polypeptide by site directed pegylation, such as by attachment to a Cys residue, where the Cys residue may be positioned at the N-terminus of the ¹⁰Fn3 polypeptide or between the N-terminus and the most N-terminal beta or beta-like strand or at the C-terminus of the ¹⁰Fn3 polypeptide or between the C-terminus and the most C-terminal beta or beta-like strand. A Cys residue may be situated at other positions as well, particularly any of the loops that do not participate in target binding. A PEG moiety may also be attached by other chemistry, including by conjugation to amines.

PEG conjugation to peptides or proteins generally involves the activation of PEG and coupling of the activated PEG-intermediates directly to target proteins/peptides or to a linker, which is subsequently activated and coupled to target proteins/peptides (see Abuchowski, A. et al, J. Biol. Chem., 252, 3571 (1977) and J. Biol. Chem., 252, 3582 (1977), Zalipsky, et al. and Harris et. al., in: Poly(ethylene glycol) Chemistry: Biotechnical and Biomedical Applications; (J. M. Harris ed.) Plenum Press: New York, 1992; Chap. 21 and 22). It is noted that a binding polypeptide containing a PEG molecule is also known as a conjugated protein, whereas the protein lacking an attached PEG molecule can be referred to as unconjugated.

A variety of molecular mass forms of PEG can be selected, e.g., from about 1,000 Daltons (Da) to 100,000 Da (n is 20 to 2300), for conjugating to binding polypeptides of the invention. The number of repeating units “n” in the PEG is approximated for the molecular mass described in Daltons. It is preferred that the combined molecular mass of PEG on an activated linker is suitable for pharmaceutical use. Thus, in one embodiment, the molecular mass of the PEG molecules does not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

In a specific embodiment of the invention, a VEGFR-2 specific inhibitor is covalently linked to one poly(ethylene glycol) group of the formula: —CO—(CH₂)ₓ—(OCH₂CH₂)ₘ—OR, with the —CO (i.e.,carbonyl) of the poly(ethylene glycol) group forming an amide bond with one of the amino groups of the binding polypeptide; R being lower alkyl; x being 2 or 3; m being from about 450 to about 950; and n and m being chosen so that the molecular weight of the conjugate minus the binding polypeptide is from about 10 to 40 kD. In one embodiment, an binding polypeptide's ε-amino group of a lysine is the available (free) amino group.

The above conjugates may be more specifically presented by formula (II): P—NHCO—(CH₂)ₓ—(OCH₂CH₂)ₘ—OR (II), wherein P is the group of a binding polypeptide as described herein, (i.e.,without the amino group or amino groups which form an amide linkage with the carbonyl shown in formula (II); and wherein R is lower alkyl; x is 2 or 3; m is from about 450 to about 950 and is chosen so that the molecular weight of the conjugate minus the binding polypeptide is from about 10 to about 40 kD. As used herein, the given ranges of “m” have an orientational meaning. The ranges of “m” are determined in any case, and exactly, by the molecular weight of the PEG group.

One skilled in the art can select a suitable molecular mass for PEG, e.g., based on how the pegylated binding polypeptide will be used therapeutically, the desired dosage, circulation time, resistance to proteolysis, immunogenicity, and other considerations. For a discussion of PEG and its use to enhance the properties of proteins, see N. V. Katre, Adv. Drug Delivery Rev. 10: 91-114 (1993).

In one embodiment of the invention, PEG molecules may be activated to react with amino groups on a binding polypeptide, such as with lysines (Bencham C. O. et al. Anal. Biochem., 131, 25 (1983); Veronese, F. M. et al. Appl. Biochem., 11, 141 (1985).; Zalipsky, S. et al. Polymeric Drugs and Drug Delivery Systems, adrs 9-110 ACS Symposium Series 469 (1999); Zalipsky, S. et al. Europ. Polym. 1, 19, 1177-1183 (1983); Delgado, C. et al. Biotechnol. Appl. Biochem., 12, 119-128 (1990)).

In one specific embodiment, carbonate esters of PEG are used to form the PEG-binding polypeptide conjugates. N,N′-disuccinimidylcarbonate (DSC) may be used in the reaction with PEG to form active mixed PEG-succinimidyl carbonate that may be subsequently reacted with a nucleophilic group of a linker or an amino group of a binding polypeptide (see U.S. Pat. Nos. 5,281,698 and 5,932,462). In a similar type of reaction, 1,1′-(dibenzotriazol yl)carbonate and di-(2-pyridyl)carbonate may be reacted with PEG to form PEG-benzotriazolyl and PEG-pyridyl mixed carbonate (U.S. Pat. No. 5,382,657), respectively.

Pegylation of a ¹⁰Fn3 polypeptide can be performed according to the methods of the state of the art, for example by reaction of the binding polypeptide with electrophilically active PEGs (supplier: Shearwater Corp., USA, www.shearwatercorp.com). Preferred PEG reagents of the present invention are, e.g., N-hydroxysuccinimidyl propionates (PEG-SPA), butanoates (PEG-SBA), PEG-succinimidyl propionate or branched N-hydroxysuccinimides such as mPEG2-NHS (Monfardini, C., et al. Bioconjugate Chem. 6 (1995) 62-69). Such methods may used to pegylated at an ε-amino group of a binding polypeptide lysine or the N-terminal amino group of the binding polypeptide.

In another embodiment, PEG molecules may be coupled to sulfhydryl groups on a binding polypeptide (Sartore, L., et al. Appl. Biochem. Biotechnol. 27, 45 (1991); Morpurgo et al. Bioconjugate Chem. 7, 363-368 (1996); Goodson et al. Bio/Technology (1990) 8, 343; U.S. Pat. No. 5,766,897). U.S. Pat. Nos. 6,610,281 and 5,766,897 describes exemplary reactive PEG species that may be coupled to sulfhydryl groups.

In some embodiments where PEG molecules are conjugated to cysteine residues on a binding polypeptide, the cysteine residues are native to the binding polypeptide, whereas in other embodiments, one or more cysteine residues are engineered into the binding polypeptide. Mutations may be introduced into an binding polypeptide coding sequence to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein. Alternatively, surface residues may be predicted by comparing the amino acid sequences of binding polypeptides, given that the crystal structure of the framework based on which binding polypeptides are designed and evolved has been solved (see Himanen et al. Nature (2001) 20-27;414(6866):933-8) and thus the surface-exposed residues identified. In one embodiment, cysteine residues are introduced into binding polypeptides at or near the N- and/or C-terminus, or within loop regions.

In some embodiments, the pegylated binding polypeptide comprises a PEG molecule covalently attached to the alpha amino group of the N-terminal amino acid. Site specific N-terminal reductive amination is described in Pepinsky et al. (2001) J. Pharmacol. Exp. Ther. 297,1059, and U.S. Pat. No. 5,824,784. The use of a PEG-aldehyde for the reductive amination of a protein utilizing other available nucleophilic amino groups is described in U.S. Pat. No. 4,002,531, in Wieder et al. (1979) J. Biol. Chem. 254,12579, and in Chamow et al. (1994) Bioconjugate Chem. 5, 133.

In another embodiment, pegylated binding polypeptide comprises one or more PEG molecules covalently attached to a linker, which in turn is attached to the alpha amino group of the amino acid residue at the N-terminus of the binding polypeptide. Such an approach is disclosed in U.S. patent application Ser. No. 09/967,223 and in PCT International Application Publication No. WO 94/01451.

In one embodiment, a binding polypeptide is pegylated at the C-terminus. In a specific embodiment, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al. Bioconjugate Chem. 2004;15(5):1005-1009.

Monopegylation of a binding polypeptide can also be produced according to the general methods described in PCT International Application Publication No. WO 94/01451. WO 94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification at the N- and/or C-terminal amino acid of the polypeptide.

The ratio of a binding polypeptide to activated PEG in the conjugation reaction can be from about 1:0.5 to 1:50, between from about 1:1 to 1:30, or from about 1:5 to 1:15. Various aqueous buffers can be used in the present method to catalyze the covalent addition of PEG to the binding polypeptide. In one embodiment, the pH of a buffer used is from about 7.0 to 9.0. In another embodiment, the pH is in a slightly basic range, e.g., from about 7.5 to 8.5. Buffers having a pKₐ close to neutral pH range may be used, e.g., phosphate buffer. Other ratios will be used when making multi-specific PEG linked proteins, such as about 1:4 to 1:8, or about 1:3 to 1:5.

Conventional separation and purification techniques known in the art can be used to purify PEGylated binding polypeptide, such as size exclusion (e.g., gel filtration) and ion exchange chromatography. Products may also be separated using SDS-PAGE. Products that may be separated include mono-, di-, tri- poly- and un-pegylated binding polypeptide, as well as free PEG. The percentage of mono-PEG conjugates can be controlled by pooling broader fractions around the elution peak to increase the percentage of mono-PEG in the composition. About ninety percent mono-PEG conjugates represents a good balance of yield and activity. Compositions in which, for example, at least ninety-two percent or at least ninety-six percent of the conjugates are mono-PEG species may be desired. In an embodiment of this invention the percentage of mono-PEG conjugates is from ninety percent to ninety-six percent.

In one embodiment, PEGylated binding polypeptide of the invention contain one, two or more PEG moieties. In one embodiment, the PEG moiety(ies) are bound to an amino acid residue which is on the surface of the protein and/or away from the surface that contacts the target ligand. In one embodiment, the combined or total molecular mass of PEG in PEG-binding polypeptide is from about 3,000 Da to 60,000 Da, optionally from about 10,000 Da to 36,000 Da. In a one embodiment, the PEG in pegylated binding polypeptide is a substantially linear, straight-chain PEG.

In one embodiment of the invention, the PEG in pegylated binding polypeptide is not hydrolyzed from the pegylated amino acid residue using a hydroxylamine assay, e.g., 450 mM hydroxylamine (pH 6.5) over 8 to 16 hours at room temperature, and is thus stable. In one embodiment, greater than 80% of the composition is stable mono-PEG-binding polypeptide, more preferably at least 90%, and most preferably at least 95%.

In another embodiment, the pegylated binding polypeptides of the invention will preferably retain at least about 25%, 50%, 60%, 70%, 80%, 85%, 90%, 95% or 100% of the biological activity associated with the unmodified protein. In one embodiment, biological activity refers to its ability to bind to VEGFR-2, as assessed by K_{d}, kₒₙ or k_{off}. In one specific embodiment, the pegylated binding polypeptide protein shows an increase in binding to VEGFR-2 relative to unpegylated binding polypeptide.

The serum clearance rate of PEG-modified polypeptide may be decreased by about 10%, 20%, 30%, 40%, 50%, 60%, 70%, 80%, or even 90%, relative to the clearance rate of the unmodified binding polypeptide. The PEG-modified polypeptide may have a half-life (t_{1/2}) which is enhanced relative to the half-life of the unmodified protein. The half-life of PEG-binding polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified binding polypeptide. In some embodiments, the protein half-life is determined in vitro, such as in a buffered saline solution or in serum. In other embodiments, the protein half-life is an in vivo half life, such as the half-life of the protein in the serum or other bodily fluid of an animal.

Additional Vectors & Polynucleotides Embodiments

Nucleic acids encoding any of the various proteins or polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See, for example: Mayfield et al. Proc. Natl. Acad. Sci. USA. 2003 Jan. 21; 100(2):438-42; Sinclair et al. Protein Expr. Purif. 2002 October; 26(1):96-105; Connell N D. Curr. Opin. Biotechnol. 2001 October; 12(5):446-9; Makrides et al. Microbiol Rev. 1996 September; 60(3):512-38; and Sharp et al. Yeast. 1991 October; 7(7):657-78.

General techniques for nucleic acid manipulation are described for example in Sambrook et al. Molecular Cloning: A Laboratory Manual, Vols. 1-3, Cold Spring Harbor Laboratory Press, 2 ed., 1989, or F. Ausubel et al. Current Protocols in Molecular Biology (Green Publishing and Wiley-Interscience: New York, 1987) and periodic updates, herein incorporated by reference. The DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The ability to replicate in a host, usually conferred by an origin of replication, and a selection gene to facilitate recognition of transformants are additionally incorporated.

The proteins may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process a native signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces .alpha.-factor leaders), or acid phosphatase leader, the C. albicans glucoamylase leader, or the signal described in PCT International Application Publication No. WO 90/13646. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2.mu. plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

In addition, vectors derived from the 1.6 .mu.m circular plasmid pKD1 can be used for transformation of Kluyveromyces yeasts. Alternatively, an expression system for large-scale production of recombinant calf chymosin was reported for K. lactis (see Van den Berg, Bio/Technology, 8:135 (1990)). Stable multi-copy expression vectors for secretion of mature recombinant human serum albumin by industrial strains of Kluyveromyces have also been disclosed (see Fleer et al. Bio/Technology, 9:968-975 (1991)).

Expression and cloning vectors usually contain a promoter that is recognized by the host organism and is operably linked to the nucleic acid encoding the protein of the invention, e.g., fibronectin based scaffolds, such as Adnectins™. Promoters suitable for use with prokaryotic hosts include the phoA promoter, beta-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the protein of the invention.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in European Pat. No. EP 73,657. Yeast enhancers also are advantageously used with yeast promoters.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al. Nature 297:598-601 (1982) on expression of human .beta.-interferon cDNA in mouse cells under the control of a thymidine kinase promoter from herpes simplex virus. Alternatively, the rous sarcoma virus long terminal repeat can be used as the promoter.

Transcription of a DNA encoding proteins of the invention by higher eukaryotes is often increased by inserting an enhancer sequence into the vector. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the multivalent antibody-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the multivalent antibody. One useful transcription termination component is the bovine growth hormone polyadenylation region (see PCT International Application Publication No. WO 94/11026 and the expression vector disclosed therein).

The recombinant DNA can also include any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts.

Additional Expression & Cell Embodiments

Preferred proteins for production and cell embodiments are fibronectin based scaffolds, such as Adnectins™, and related proteins.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for protein-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichodenna reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated proteins of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

In some instance it will be desired to produce proteins in vertebrate cells, such as glycosylation, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al. J. Gen Virol. 36:59. (1977)); baby hamster kidney cells (BHK, ATCC CCL 10); Chinese hamster ovary cells/-DHFR (CHO, Urlaub et al. Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al. Annals N.Y. Acad. Sci. 383:44-68 (1982)); MRC 5 cells; FS4 cells; a human hepatoma line (Hep G2); and myeloma or lymphoma cells (e.g., Y0, J558L, P3 and NS0 cells) (see U.S. Pat. No. 5,807,715).

Plant cell cultures of cotton, corn, potato, soybean, petunia, tomato, and tobacco can also be utilized as hosts.

Host cells are transformed with the herein-described expression or cloning vectors for protein production and cultured in conventional nutrient media modified as appropriate for inducing promoters, selecting transformants, or amplifying the genes encoding the desired sequences.

Culturing Cells

The host cells used to produce the proteins of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al. Meth. Enz. 58:44 (1979), Barnes et al. Anal. Biochem.102:255 (1980), U.S. Pat. Nos. 4,767,704; 4,657,866; 4,927,762; 4,560,655; or 5,122,469; published PCT International Application Publication Nos. WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Proteins of the invention can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

The proteins of the present invention can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, more preferably at least 95% pure, and most preferably at least 98% pure. Regardless of the exact numerical value of the purity, the polypeptide is sufficiently pure for use as a pharmaceutical product.

Additional Glycosylation Embodiments

In some embodiments it may be preferable to glycosylate proteins used for the invention. Preferably, such proteins are fibronectin based scaffolds, such as Adnectins™. Adnectins™ do not normally contain glycosylation sites, however, such glycosylation may be engineered into the protein.

Glycosylation of proteins is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. These can be engineered into the proteins of the invention, in particular fibronectin based scaffolds, such as Adnectins™, and their corresponding polynucleotides. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the proteins of the invention are conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding such amino acid sequence variants of the proteins of the invention are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the protein (e.g., fibronectin based scaffolds, such as Adnectins™).

It may be desirable to modify proteins of the invention with respect to effector function, e.g., so as to enhance antigen-dependent cell-mediated cyotoxicity (ADCC) and/or complement dependent cytotoxicity (CDC) of the antibody. This may be achieved by introducing an active portion of an Fc region, as well as one or more amino acid modifications in an Fc region of the protein (e.g., fibronectin based scaffolds, such as Adnectins™,), thereby generating a variant Fc region. The Fc region variant may comprise a human Fc region sequence (e.g., a human IgG1, IgG2, IgG3 or IgG4 Fc region) comprising an amino acid modification (e.g., a substitution) at one or more amino acid positions.

In one embodiment, the variant Fc region may mediate antibody-dependent cell-mediated cytotoxicity (ADCC) in the presence of human effector cells more effectively, or bind an Fc gamma receptor (FcγR) with better affinity, than a native sequence Fc region. Such Fc region variants may comprise an amino acid modification at any one or more of positions 256, 290, 298, 312, 326, 330, 333, 334, 360, 378 or 430 of the Fc region, wherein the numbering of the residues in the Fc region is that of the EU index as in Kabat.

Additional Antibody Based Proteins, Including Moieties and Derivatives

Additional embodiments of proteins useful for the invention, include single domain antibodies, antibodies whose complementary determining regions are part of a single domain polypeptide, preferably, directed to VEGFR-2, as well as included in the PEG linked proteins of invention. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one aspect of the invention, a single domain antibodies as used herein is a naturally occurring single domain antibody known as heavy chain antibody devoid of light chains. Such single domain antibodies are disclosed, for example, in PCT International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, dromedary, llama, vicuna, alpaca and guanaco. Other species besides Camelidae may produce heavy chain antibodies naturally devoid of light chain; such VHHs are within the scope of the invention. VHHs, according to the present invention, and as known to the skilled in the art, are heavy chain variable domains derived from immunoglobulins naturally devoid of light chains such as those derived from Camelidae as described in PCT International Application Publication No. WO 94/04678 (and referred to hereinafter as VHH domains or nanobodies). VHH molecules are about 10 times smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognized by antibodies generated in vitro through the use of antibody libraries or via immunisation of mammals other than Camelids (see PCT International Application Publication No. WO 9749805). As such, anti-VEGFR-2 VHH's may interact more efficiently with VEGFR-2 than conventional antibodies, thereby blocking its interaction with the VEGFR ligand(s) more efficiently. Since VHH's are known to bind into ‘unusual’ epitopes such as cavities or grooves (see PCT International Application Publication No. WO 97/49805), the affinity of such VHH's may be more suitable for therapeutic treatment.

Another example of a VEGFR-2 specific inhibitor useful in the methods of the invention is an anti-VEGFR-2 consisting of a sequence corresponding to that of a Camelidae VHH directed towards VEGFR-2 or a closely related family member. The invention also relates to a homologous sequence, a function portion or a functional portion of a homologous sequence of said polypeptide. The invention also relates to nucleic acids capable of encoding said polypeptides. A single domain antibody of the present invention may be directed against VEGFR-2 or a closely related family member.

A polypeptide construct useful in methods of the invention may further comprise single domain antibodies directed against other targets such as, for example, serum albumin. A single domain antibody directed against a target means a single domain antibody that is capable of binding to said target with an affinity of better than 10⁻⁶ M.

The present invention further relates to the use of single domain antibodies of VHH belonging to a class having human-like sequences. One such class is characterized in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 and a tryptophan at position 103, according to the Kabat numbering. As such, polypeptides belonging to this class show a high amino acid sequence homology to human VH framework regions and said polypeptides might be administered to a human directly without expectation of an unwanted immune response therefrom, and without the burden of further humanisation.

Another human-like class of Camelidae single domain antibodies has been described in PCT International Application Publication No. WO 03/035694 and contain the hydrophobic FR2 (framework 2) residues typically found in conventional antibodies of human origin or from other species, but compensating this loss in hydrophilicity by the charged arginine residue on position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanization. The invention also relates to nucleic acids capable of encoding said polypeptides.

Polypeptides may include the full length Camelidae antibodies, namely Fc and VHH domains.

Anti-albumin VHH's may interact in a more efficient way with serum albumin which is known to be a carrier protein. As a carrier protein some of the epitopes of serum albumin may be inaccessible by bound proteins, peptides and small chemical compounds. Since VHH's are known to bind into ‘unusual’ or non-conventional epitopes such as cavities (see PCT International Application Publication No. WO 97/49805), the affinity of such VHH's to circulating albumin may be more suitable for therapeutic treatment. The serum protein may be any suitable protein found in the serum of subject, or fragment thereof. In one aspect of the invention, the serum protein is serum albumin, serum immunoglobulins, thyroxine-binding protein, transferrin, or fibrinogen. Depending on the intended use such as the required half-life for effective treatment and/or compartimentalisation of the target antigen, the VHH-partner can be directed to one of the above serum proteins.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al. Nature 341, 544-546 (1989)) which consists of a VH domain; (vii) isolated CDR regions; (viii) F(ab′)₂ fragments, a bivalent fragment including two Fab' fragments linked by a disulphide bridge at the hinge region; (ix) single chain antibody molecules (e.g., single chain Fv; scFv) (Bird et al. Science 242:423-426 (1988); and Huston et al. Proc. Natl. Acad. Sci. USA 85:5879-5883 (1988)); (x) “diabodies” with two antigen binding sites, comprising a heavy chain variable domain (VH) connected to a light chain variable domain (VL) in the same polypeptide chain (see, e.g., European Pat. No. EP 404,097; PCT International Application Publication No. WO 93/11161; and Hollinger et al. Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

Various techniques have been developed for the production of antibody fragments that may be used to make antibody fragments used in the invention. Traditionally, these fragments were derived via proteolytic digestion of intact antibodies (see, e.g., Morimoto et al. J. Biochem. Bioph. Methods 24:107-117 (1992); and Brennan et al. Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv) (See PCT International Application Publication No. WO 93/16185 and U.S. Pat. Nos. 5,571,894 and 5,587,458). The antibody fragment may also be a “linear antibody”, e.g., as described in U.S. Pat. No. 5,641,870, for example. Such linear antibody fragments may be monospecific or bispecific.

Alternative Targeted Protein Therapeutics

In certain embodiments, the proteins useful in methods of the invention described herein may comprise one or more avimer sequences. Avimers are a type of binding proteins that have affinities and specificities for various target molecules, including those described herein. These proteins can be included in the PEG linked embodiments of the invention. They were developed from human extracellular receptor domains by in vitro exon shuffling and phage display (see Silverman et al. 2005, Nat. Biotechnol. 23:1493-94; and Silverman et al. 2006, Nat. Biotechnol. 24:220.) The resulting multidomain proteins may comprise multiple independent binding domains, that may exhibit improved affinity (in some cases sub-nanomolar) and specificity compared with single-epitope binding proteins. In various embodiments, avimers may be attached to, for example, with PEG or polpeptide linkers. Additional details concerning methods of construction and use of avimers are disclosed, for example, in U.S. patent application Ser. Nos. 10/693,057, 10/693,056, 10/840,723, 10/871,602 and 10/966,064, the Examples section of each of which is incorporated herein by reference, as well as the entire document.

In certain embodiments, the proteins useful in the methods of the invention described herein may comprise one or more lipocalin related sequences, e.g., anticalins or lipocalin derivatives. Anticalins or lipocalin derivatives are a type of binding proteins that have affinities and specificities for various target molecules, including those described herein. Such proteins are described in U.S. patent application Ser. No. 11/224,071—Anticalins, U.S. patent application Ser. No. 10/490,953—“Muteins of human neutrophil gelatinase-associated lipocalin and related proteins”, U.S. patent application Ser. No. 10/491,001—“Muteins of apolipoprotein d” and PCT International Application Publication No. WO 06/056464. These proteins can be included in the PEG linked embodiments of the invention.

In certain embodiments, the proteins useful in the methods of the invention described herein may comprise one or more tetranectin C-type lectin related sequences or trinectins, e.g., tetranectin C-type lectin or tetranectin C-type lectin derivatives. Tetranectin C-type lectins or tetranectin C-type lectin derivatives are a type of binding proteins that have affinities and specifities for various target molecules including those described herein. Different tetranectin C-type lectin and related proteins are described in PCT International Application Publication Nos WO 06/053568; WO 05/080418; WO 04/094478; WO 04/039841; WO 04/005335; WO 02/048189; and WO 98/056906, and U.S. patent application Ser. No 11/064,115. These proteins can be included in the PEG linked embodiments of the invention.

Toxins and Other Molecules Linked to Proteins of the Invention

The proteins useful in the methods of the invention as disclosed herein, may be linked to a cytotoxic agent. Such embodiments can be prepared by in vitro or in vivo methods as appropriate.

In vitro methods, include conjugation chemistry well know in the art including chemistry compatible with proteins, such as chemistry for specific amino acids, such as Cys and Lys. In order to link a cytotoxic agent to protein of the invention, a linking group or reactive group is used. Suitable linking groups are well known in the art and include disulfide groups, thioether groups, acid labile groups, photolabile groups, peptidase labile groups and esterase labile groups. Preferred linking groups are disulfide groups and thioether groups. For example, conjugates can be constructed using a disulfide exchange reaction or by forming a thioether bond between the antibody and the cytotoxic agent. Preferred cytotoxic agents are maytansinoids, taxanes and analogs of CC-1065.

In vivo methods include linking toxic, tagging or labeling proteins to proteins of the invention as fusion proteins. A single polypeptide is produced using an encoding polynucleotide for the desired polypeptide. Toxic proteins can be controlled by expressing in toxin resistant or insensitive cells or with inducible promoters in cells that are sensitive.

Although not limiting, in various embodiments, proteins of the invention may be linked to proteins, such as a bacterial toxin, a plant toxin, ricin, abrin, a ribonuclease (RNase), DNase I, a protease, Staphylococcal enterotoxin-A, pokeweed antiviral protein, gelonin, diphtherin toxin, Pseudomonas exotoxin, Pseudomonas endotoxin, Ranpimase (Rap), Rap (N69Q), an enzyme, or a fluorescent protein.

Maytansinoids and maytansinoid analogs are among the preferred cytotoxic agents. Examples of suitable maytansinoids include maytansinol and maytansinol analogs. Suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Taxanes are also preferred cytotoxic agents. Taxanes suitable for use in the present invention are disclosed in U.S. Pat. Nos. 6,372,738 and 6,340,701.

CC-1065 and its analogs are also preferred cytotoxic drugs for use in the present invention. CC-1065 and its analogs are disclosed in U.S. Pat. Nos. 6,372,738; 6,340,701; 5,846,545 and 5,585,499.

An attractive candidate for the preparation of such cytotoxic conjugates is CC-1065, which is a potent anti-tumor antibiotic isolated from the culture broth of Streptomyces zelensis. CC-1065 is about 1000-fold more potent in vitro than are commonly used anti-cancer drugs, such as doxorubicin, methotrexate and vincristine (B. K. Bhuyan et al. Cancer Res., 42, 3532-3537 (1982)).

Cytotoxic drugs such as methotrexate, daunorubicin, doxorubicin, vincristine, vinblastine, melphalan, mitomycin C, chlorambucil, and calicheamicin are also suitable for the preparation of conjugates of the present invention, and the drug molecules can also be linked to the antibody molecules through an intermediary carrier molecule such as serum albumin.

Conjugation

Any method known in the art for conjugating the a protein to the detectable moiety may be employed, including those methods described by Hunter, et al. Nature 144:945 (1962); David, et al. Biochemistry 13:1014 (1974); Pain, et al. J. Immunol. Meth. 40:219 (1981); and Nygren, J. Histochem. and Cytochem. 30:407 (1982).

In vitro methods, include conjugation chemistry well know in the art including chemistry compatible with proteins, such as chemistry for specific amino acids, such as Cys and Lys. In order to link a moiety (such as PEG) to a protein of the invention, a linking group or reactive group is used. Suitable linking groups are well known in the art and include disulfide groups, thioether groups, acid labile groups, photolabile groups, peptidase labile groups and esterase labile groups. Preferred linking groups are disulfide groups and thioether groups depending on the application.

For fibronectin based scaffolds, such as Adnectins™, or other proteins with out a Cys amino acid, a Cys can be engineered in a location to allow for activity of the protein to exist while creating a location for conjugation.

Formulation and Administration

Therapeutic formulations of the invention are prepared for storage by mixing the described proteins having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations herein may also contain more than one active compound as necessary for the particular indication being treated, preferably those with complementary activities that do not adversely affect each other. Examples of combinations of active compounds are provided in herein. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the proteins of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins of the invention may remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

While the skilled artisan will understand that the dosage of each therapeutic agent will be dependent on the identity of the agent, the preferred dosages can range from about 10 mg/square meter to about 2000 mg/square meter, more preferably from about 50 mg/square meter to about 1000 mg/square meter. For preferred agents such as platinum agents (carboplatin, oxaliplatin, cisplatin), the preferred dosage is about 10 mg/square meter to about 400 mg/square meter, for taxanes (paclitaxel, docetaxel) the preferred dosage is about 20 mg/square meter to about 150 mg/square meter, for gemcitabine the preferred dosage is about 100 mg/square meter to about 2000 mg/square meter, and for camptothecin the preferred dosage is about 50 mg/square meter to about 350 mg/square meter. The dosage of this and other therapeutic agents may depend on whether the antibody, antibody fragment or conjugate of the invention is administered concurrently or sequentially with a therapeutic agent.

The VEGFR-2 specific inhibitors are administered to a subject in a pharmaceutically acceptable dosage form. They can be administered intravenously as a bolus or by continuous infusion over a period of time, by intramuscular, subcutaneous, intra-articular, intrasynovial, intrathecal, intraocular, oral, topical, or inhalation routes. The protein may also be administered by intratumoral, peritumoral, intralesional, or perilesional routes, to exert local as well as systemic therapeutic effects. Suitable pharmaceutically acceptable carriers, diluents, and excipients are well known and can be determined by those of skill in the art as the clinical situation warrants. Examples of suitable carriers, diluents and/or excipients include: (1) Dulbecco's phosphate buffered saline, pH about 7.4, containing about 1 mg/mL to 25 mg/mL human serum albumin, (2) 0.9% saline (0.9% w/v NaCl), and (3) 5% (w/v) dextrose. The method of the present invention can be practiced in vitro, in vivo, or ex vivo. Examples of fonnulations include 5-100 mM sodium acetate, mannitol or a comparable excipient (e.g., sorbitol) at concentrations from 50 mM to and including hypertonic concentrations, optionally sodium chloride from 0 to about 200 mM, with a pH from about 4 about 7.5. In one embodiment, the formulation comprises around 10 mM sodium acetate, around 100 mM sodium chloride, and around 110 mM mannitol with a pH of 4.5. In another embodiment, the formulation comprises 10 mM sodium acetate and 100 mM of mannitol at a pH from about 4.5 to about 6. The inhibitors can be in the formulation in a concentration of from 1 to 15 mg/mL. In one embodiment the concentration of the protein is from about 9 to about 11 mg/mL. Administration of a VEGFR-2 specific inhibitor, and one or more additional therapeutic agents, whether co-administered or administered sequentially, may occur as described above. Suitable pharmaceutically acceptable carriers, diluents, and excipients for co-administration will be understood by the skilled artisan to depend on the identity of the particular therapeutic agent being co-administered.

When present in an aqueous dosage form, rather than being lyophilized, the protein typically will be formulated at a concentration of about 0.1 mg/mL to about 100 mg/mL, although wide variation outside of these ranges is permitted. For the treatment of disease, the appropriate dosage of antibody or conjugate will depend on the type of disease to be treated, as defined above, the severity and course of the disease, whether the antibodies are administered for preventive or therapeutic purposes, the course of previous therapy, the patient's clinical history and response to the antibody, and the discretion of the attending physician. The protein is suitably administered to the patient at one time or over a series of treatments.

Depending on the type and severity of the disease, preferably from about 1 mg/square meter to about 2000 mg/square meter of protein is an initial candidate dosage for administration to the patient, more preferably from about 10 mg/square meter to about 1000 mg/square meter of antibody whether, for example, by one or more separate administrations, or by continuous infusion. For repeated administrations over several days or longer, depending on the condition, the treatment is repeated until a desired suppression of disease symptoms occurs. However, other dosage regimens may be useful and are not excluded.

The present invention also includes kits useful in treating patients having or at risk of developing metastatic cancer comprising one or more of the elements described herein, and instructions for the use of those elements. In a preferred embodiment, a kit of the present invention includes a VEGFR-2 specific inhibitor and an anti-neoplastic composition. The anti-neoplastic composition used in the kit may contain any suitable chemotherapeutic as previously described herein for any cancer as previously described herein.

When a kit is supplied, the different components of the composition may be packaged in separate containers and admixed immediately before use. Such packaging of the components separately may permit long-term storage without losing the active components' functions.

The reagents included in the kits can be supplied in containers of any sort such that the life of the different components are preserved and are not adsorbed or altered by the materials of the container. For example, sealed glass ampules may contain lyophilized therapeutic agents, or buffers that have been packaged under a neutral, non-reacting gas, such as nitrogen. Ampules may consist of any suitable material, such as glass, organic polymers, such as polycarbonate, polystyrene, etc., ceramic, metal or any other material typically employed to hold similar reagents. Other examples of suitable containers include simple bottles that may be fabricated from similar substances as ampules, and envelopes, that may comprise foil-lined interiors, such as aluminum or an alloy. Other containers include test tubes, vials, flasks, bottles, IV bags, syringes, or the like. Containers may have a sterile access port, such as a bottle having a stopper that can be pierced by a hypodermic injection needle. Other containers may have two compartments that are separated by a readily removable membrane that upon removal permits the components to be mixed. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, flash memory device etc. Detailed instructions may not be physically associated with the kit; instead, a user may be directed to an internet web site specified by the manufacturer or distributor of the kit, or supplied as electronic mail.

The dosage of cytotoxic or therapeutic agents administered in the methods described herein can be readily determined by those skilled in the art. Pharmaceutical package inserts may also be consulted when determining the proper dosage. By way of example, the following package inserts are obtainable on the world wide web at:
pfizer.com/pfizer/download/news/asco/sutent_fact_sheet.pdf, for Sutent™: univgraph.com/bayer/inserts/nexavar.pdf, for Nexavar™;
gene.com/gene/products/information/oncology/bevacizumab/insert.jsp, for bevacizumab;
gene.com/gene/products/information/oncology/herceptin/index.jsp, for trastuzumab;
pi.lilly.com/us/gemzar.pdf, for gemcitabine;
fda.gov/cder/foi/label/1999/21029lbl.pdf, for temozolomide;
accessdata.fda.gov/scripts/cder/onctools/labels.cfm?GN=imatinib%20mesylate, for Gleevac™;
accessdata.fda.gov/scripts/cder/onctools/labels.cfm?GN=paclitaxel, for paclitaxel;
http://www.accessdata.fda.gov/scripts/cder/onctools/labels.cfm?GN=docetaxel, for doxetaxel;
and are incorporated by reference.

Additional Patent References

Methods and compositions described in the following additional Patent Applications and Patents are also included in this disclosure: U.S. patent application Ser. Nos. 10/897,406; 10/735,916; 10/886,838; 10/800,197; 10/363,552; 10/309,722; 11/259,232; 11/154,103; 10/553,105; 10/650,592; 10/650,591; 10/792,498; 10/676,873; 10/728,078; 10/989,723; 11/483,918; 11/448,171; and 11/482,641; U.S. Pat. Nos. 5,707,632; 6,818,418; and 7,115,396; and PCT International Application Publication Nos. WO 05/085430; WO 04/019878; WO 04/029224; WO 05/056764; WO 01/064942; and WO 02/032925.

Incorporation by Reference

All documents and references, including patent documents and websites, described herein are individually incorporated by reference to into this document to the same extent as if there were written in this document in full or in part.

The invention is now described by reference to the following examples, which are illustrative only, and are not intended to limit the present invention. While the invention has been described in detail and with reference to specific embodiments thereof, it will be apparent to one of skill in the art that various changes and modifications can be made thereto without departing from the spirit and scope thereof.

### Examples

### Example 1

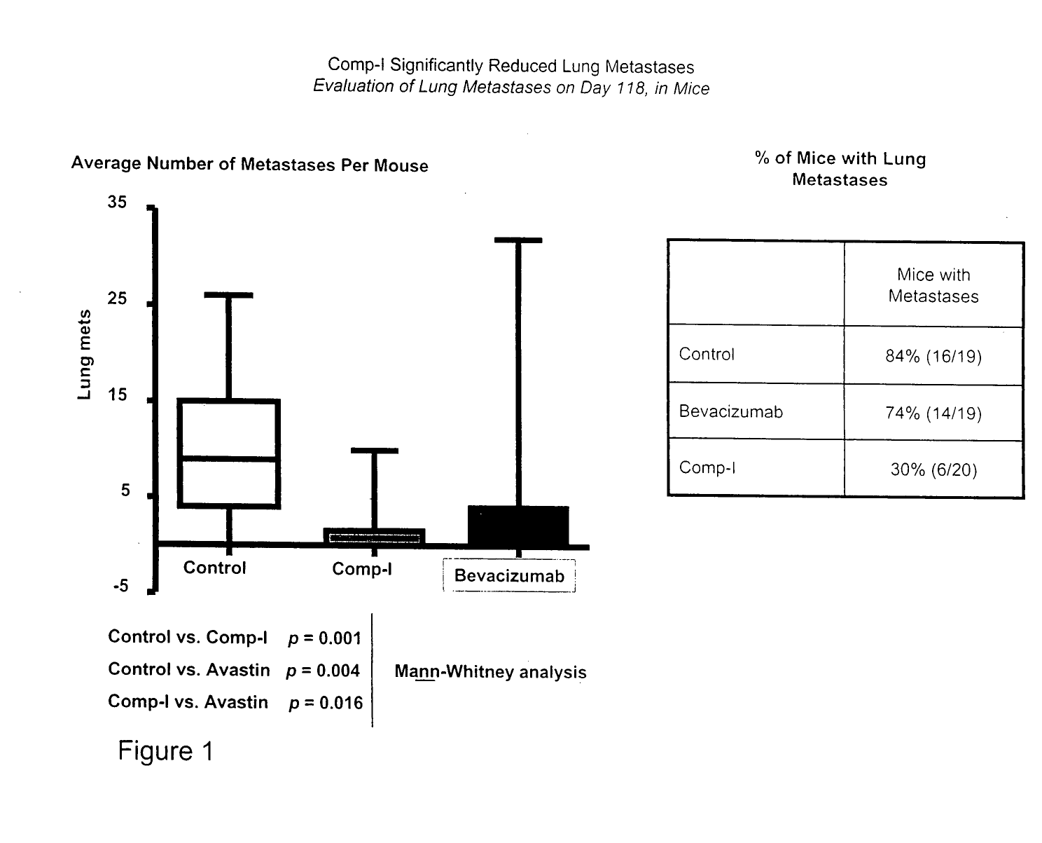

In an orthotopic model of tumor metastasis, Comp-I was substantially more active than bevacizumab. Human MDA-MB-231 breast cancer cells were implanted in the mouse mammary fat pads at day 0 (1×10⁶ MDA-MB-231 breast cancer cells). The resultant tumors were resected at day 24 and treatment initiated 4 days later (Mean tumor volume at resection=400 mm³). Treatment groups were as follows: Control: PBS, Comp-I: 30 mg/kg, and bevacizumab 5 mg/kg (100 μL IP injections twice weekly). When mice were sacrificed, the extent of local regrowth and numbers of lung metastases were evaluated. Comp-I produced fewer macroscopic metastases per mouse than bevacizumab. Furthermore, Comp-I had a remarkable effect on the incidence of metastases in the study. Only 30% of Comp-1-treated mice showed lung metastases compared with 74% of bevacizumab-treated animals and 84% of the control cohort (see FIG. 1).

Sequence Listing

SEQ ID NO:1 is the tenth module of the human fibronectin type III domain.

SEQ ID NOs:2-60 are VEGFR-2 binding ¹⁰Fn3 polypeptides.

SEQ ID NO:61 is a C-terminal tail suitable for pegylation.

SEQ ID NOs:62-65 are fragments, specifically loop structures of the tenth module of the human fibronectin type III domain.

1. A method of treating, preventing, or reducing the spread of metastatic cancer, the method comprising administering, to a patient in need thereof, a therapeutic VEGFR-2 specific inhibitor.

2. The method of claim 1, wherein the patient is afflicted with breast cancer.

3. The method of claim 1, wherein the VEGFR-2 specific inhibitor comprises a first polypeptide that binds to human VEGFR-2, the polypeptide comprising between about 80 and about 150 amino acids that have a structural organization comprising: a) at least five to seven beta strands or beta-like strands distributed among at least two beta sheets, and b) at least one loop portion connecting two strands that are beta strands or beta-like strands, which loop portion participates in binding to VEGFR-2, wherein the polypeptide binds to an extracellular domain of the human VEGFR-2 protein with a dissociation constant (K_{d}) of less than 1×10⁻⁶ M and inhibits VEGFR-2 mediated angiogenesis.

4. The method of claim 3, wherein the polypeptide comprises an amino acid sequence that is at least 80% identical to SEQ NO:1.

5. The method of claim 3, wherein the polypeptide comprises an amino acid sequence selected from the group consisting of any of SEQ ID NOs:2-60.

6. The method of claim 3, wherein the polypeptide further comprises a polyoxyalkylene moiety.

7. The method of claim 6, wherein the polyoxyalkylene moiety is a polyethylene glycol (PEG) moiety.

8. The method of claim 1, further comprising administration of a second chemotherapeutic agent.

9. The method of claim 8, wherein said second agent is sunitinib malate.

10. The method of claim 8, wherein said second agent is lapatinib.

11. The method of claim 8, wherein said second agent is sorafenib.

12. The method of claim 8, wherein said second agent is AZD2171.

13. The method of claim 8, wherein said second agent is bevacizumab.

14. The method of claim 8, wherein said second agent is aflibercept.

15. The method of claim 8, wherein said second agent is an mTor inhibitor.

16. The method of claim 15, wherein the mTor inhibitor is rapamycin.

17. The method of claim 8, wherein said second agent is gemcitabine.

18. The method of claim 8, wherein said second agent is temozolomide.

19. The method of claim 8, wherein said second agent is dastinib.

20. The method of claim 8, wherein said second agent is cetuximab.

21. The method of claim 8, wherein said second agent is temsirolimus.

22. The method of claim 8, wherein said second agent is ixabepilone.

23. The method of claim 8, wherein said second agent is imatinib mesylate.

24. The method of claim 8, wherein said second agent is trastuzumab.

25. The method of claim 8, wherein said second agent is a taxane.

26. The method of claim 8, wherein the second agent is oxaliplatin.

27. The method of claim 8, wherein the second agent is 5-fluorouracil

28. The method of claim 3, wherein the VEGFR-2 specific inhibitor further comprises a second polypeptide, the polypeptide comprising an amino acid sequence that is at least 80% identical to SEQ NO:1.


SEQUENCE LISTING (65 sequences)

SEQ ID NO: 1 - protein, 94 aa, Homo sapiens
VSDVPRDLEVVAATPTSLLISWDAPAVTVRYYRITYGETGGNSPVQEFTVPGSKSTATISGLKPGVDYTITGYAVTGRGDSPASSKPISINYRT

SEQ ID NO: 2 - protein, 94 aa, Artificial Sequence
  Description of Artificial Sequence: Synthetic polypeptide
VSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 3 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITVYAVTEGPNERSLFIPISINYRT

SEQ ID NO: 4 - protein, 94 aa, Artificial Sequence
  Description of Artificial Sequence: Synthetic polypeptide
GEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 5 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITVYAVTDGRNGRLLSIPISINYRT

SEQ ID NO: 6 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTMGLYGHELLTPISINYRT

SEQ ID NO: 7 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTDGENGQFLLVPISINYRT

SEQ ID NO: 8 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTMGPNDNELLTPISINYRT

SEQ ID NO: 9 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTAGWDDHELFIPISINYRT

SEQ ID NO: 10 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTSGHNDHMLMIPISINYRT

SEQ ID NO: 11 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTAGYNDQILMTPISINYRT

SEQ ID NO: 12 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTFGLYGKELLIPISINYRT

SEQ ID NO: 13 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTTGPNDRLLFVPISINYRT

SEQ ID NO: 14 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTDVYNDHEIKTPISINYRT

SEQ ID NO: 15 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTDGKDGRVLLTPISINYRT

SEQ ID NO: 16 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTEVHHDREIKTPISINYRT

SEQ ID NO: 17 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTQAPNDRVLYTPISINYRT

SEQ ID NO: 18 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTREENDHELLIPISINYRT

SEQ ID NO: 19 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVTHNGHPLMTPISINYRT

SEQ ID NO: 20 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTLALKGHELLTPISINYRT

SEQ ID NO: 21 - protein, 94 aa, Artificial Sequence
  Description of Artificial Sequence: Synthetic polypeptide
VSDVPRDLEVVAATPTSLLISWRHPHFPTRYYRITYGETGGNSPVQEFTVPLQPPTATISGLKPGVDYTITGYAVTVAQNDHELITPISINYRT

SEQ ID NO: 22 - protein, 95 aa, Artificial Sequence
  Description of Artificial Sequence: Synthetic polypeptide
VSDVPRDLQEVVAATPTSLLISWRHPHFPTRYYRITYGETGGNSPVQEFTVPLQPPAATISGLKPGVDYTITGYAVTMAQSGHELFTPISINYRT

SEQ ID NO: 23 - skipped

SEQ ID NO: 24 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVERNGRVLMTPISINYRT

SEQ ID NO: 25 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVERNGRHLMTPISINYRT

SEQ ID NO: 26 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTLERNGRELMTPISINYRT

SEQ ID NO: 27 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTEERNGRTLRTPISINYRT

SEQ ID NO: 28 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVERNDRVLFTPISINYRT

SEQ ID NO: 29 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVERNGRELMTPISINYRT

SEQ ID NO: 30 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTLERNGRELMVPISINYRT

SEQ ID NO: 31 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTDGRNDRKLMVPISINYRT

SEQ ID NO: 32 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTDGQNGRLLNVPISINYRT

SEQ ID NO: 33 - protein, 87 aa, Artificial Sequence
  Description of Artificial Sequence: Synthetic polypeptide
EVVAATPTSLLISWRHHPHFPTRYYRITYGETGGNSPVQEFTVPLQPPTATISGLKPGVDYTITGYAVTVHWNGRELMTPISINYRT

SEQ ID NO: 34 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTEEWNGRVLMTPISINYRT

SEQ ID NO: 35 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVERNGHTLMTPISINYRT

SEQ ID NO: 36 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVEENGRQLMTPISINYRT

SEQ ID NO: 37 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTLERNGQVLFTPISINYRT

SEQ ID NO: 38 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVERNGQVLYTPISINYRT

SEQ ID NO: 39 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTWGYKDHELLIPISINYRT

SEQ ID NO: 40 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTLGRNDRELLTPISINYRT

SEQ ID NO: 41 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTDGPNDRLLNIPISINYRT

SEQ ID NO: 42 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTFARDGHEILTPISINYRT

SEQ ID NO: 43 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTLEQNGRELMTPISINYRT

SEQ ID NO: 44 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTVEENGRVLNTPISINYRT

SEQ ID NO: 45 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTLEPNGRYLMVPISINYRT

SEQ ID NO: 46 - protein, 86 aa, Artificial Sequence
  Description of Artificial Sequence: Synthetic polypeptide
EVVAATPTSLLISWRHPHFPTRYYRITYGETGGNSPVQEFTVPLQPPTATISGLKPGVDYTITGYAVTEGRNGRELFIPISINYRT

SEQ ID NO: 47 - protein, 94 aa, Artificial Sequence
  Description of Artificial Sequence: Synthetic polypeptide
VSDVPRDLEVVAATPTSLLISWRHPHFPTRYYRITYGETGGNSPVQEFTVPLQPPAATISGLKPGVDYTITGYAVTWERNGRELFTPISINYRT

SEQ ID NO: 48 - protein, 94 aa, Artificial Sequence
  Description of Artificial Sequence: Synthetic polypeptide
VSDVPRDLEVVAATPTSLLISWRHPHFPTRYYRITYGETGGNSPVQEFTVPLQPPAATISGLKPGVDYTITGYAVTKERNGRELFTPISINYRT

SEQ ID NO: 49 - protein, 94 aa, Artificial Sequence
  Description of Artificial Sequence: Synthetic polypeptide
VSDVPRDLEVVAATPTSLLISWRHPHFPTHYYRITYGETGGNSPVQEFTVPLQPPAATISGLKPGVDYTITGYAVTTERTGRELFTPISINYRT

SEQ ID NO: 50 - protein, 94 aa, Artificial Sequence
  Description of Artificial Sequence: Synthetic polypeptide
VSDVPRDLEVVAATPTSLLISWRHPHFPTHYYRITYGETGGNSPVQEFTVPLQPPAATISGLKPGVDYTITGYAVTKERSGRELFTPISINYRT

SEQ ID NO: 51 - protein, 94 aa, Artificial Sequence
  Description of Artificial Sequence: Synthetic polypeptide
VSDVPRDLEVVAATPTSLLISWRHPHFPTHYYRITYGETGGNSPVQEFTVPLQPPAATISGLKPGVDYTITGYAVTLERDGRELFTPISINYRT

SEQ ID NO: 52 - protein, 95 aa, Artificial Sequence
  Description of Artificial Sequence: Synthetic polypeptide
VSDVPRDLEVVAATPTSLLISWRHPHFPTRYYRITYGETGGNSPVQEFTVPLQPPLATISGLKPGVDYTITGVYAVTKERNGRELFTPISINYRT

SEQ ID NO: 53 - protein, 94 aa, Artificial Sequence
  Description of Artificial Sequence: Synthetic polypeptide
VSDVPRDLEVVAATPTSLLISWRHPHFPTRYYRITYGETGGNSPVQEFTVPLQPTTATISGLKPGVDYTITGYAVTWERNGRELFTPISINYRT

SEQ ID NO: 54 - protein, 94 aa, Artificial Sequence
  Description of Artificial Sequence: Synthetic polypeptide
VSDVPRDLEVVAATPTSLLISWRHPHFPTRYYRITYGETGGNSPVQEFTVPLQPTVATISGLKPGVDYTITGYAVTLERNDRELFTPISINYRT

SEQ ID NO: 55 - protein, 95 aa, Artificial Sequence
  Description of Artificial Sequence: Synthetic polypeptide
MGEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 56 - protein, 95 aa, Artificial Sequence
  Description of Artificial Sequence: Synthetic polypeptide
MGEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 57 - protein, 102 aa, Artificial Sequence
  Description of Artificial Sequence: Synthetic polypeptide
MVSDVPRDLEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 58 - protein, 88 aa, Artificial Sequence
  Description of Artificial Sequence: Synthetic polypeptide
MGEVVAATPTSLLISWRHPHFPTRYYRITYGETGGNSPVQEFTVPLQPPTATISGLKPGVDYTITVYAVTDGWNGRLLSIPISINYRT

SEQ ID NO: 59 - protein, 88 aa, Artificial Sequence
  Description of Artificial Sequence: Synthetic polypeptide
MGEVVAATPTSLLISWRHPHFPTRYYRITYGETGGNSPVQEFTVPLQPPTATISGLKPGVDYTITVYAVTEGPNERSLFIPISINYRT

SEQ ID NO: 60 - protein, 95 aa, Artificial Sequence
  Description of Artificial Sequence: Synthetic polypeptide
MVSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 61 - protein, 7 aa, Artificial Sequence
  Description of Artificial Sequence: Synthetic peptide
EIDKPCQ

SEQ ID NO: 62 - protein, 5 aa, Homo sapiens
VSDVP

SEQ ID NO: 63 - protein, 8 aa, Homo sapiens
DAPAVTVR

SEQ ID NO: 64 - protein, 5 aa, Homo sapiens
GSKST

SEQ ID NO: 65 - protein, 11 aa, Homo sapiens
GRGDSPASSKP